(19) United States
(12) Patent Application Publication
(10) Pub. No.: US 2026/0204389 A1
(43) Pub. Date: Jul. 16, 2026

(54) SYSTEM FOR REPRESENTING A QUALITY AND HEALTH OF AN INDIVIDUAL MICROBIOME

(71) Applicant: Zoe Limited, London (GB)

(72) Inventors: Jonathan Thomas Wolf, London (GB); Laura Anne Mahy, London (GB); Marc Heinz Dietrich Lindner, London (GB)

(21) Appl. No.: 19/441,999

(22) Filed: Jan. 7, 2026

Related U.S. Application Data
(60) Provisional application No. 63/744,502, filed on Jan. 13, 2025.

(51) Int. Cl. G16H 20/60; G06T 11/00; G16H 50/30

(52) U.S. Cl. CPC G16H 20/60; G06T 11/00; G16H 50/30

(57) ABSTRACT

The present application provides methods and systems for visualizing a quality and health of an individual's microbiome. In some examples, the system represents the microbiome of the individual as an avatar or virtual character that illustrates attributes of the individual's personal microbiome health based at least in part on the individual's actual nutritional consumption and participant data representing data associated with the individual's microbiome.

### CROSS-REFERENCE TO RELATED APPLICATIONS

This application claims priority to U.S. Provisional Application No. 63/744,502 filed on Jan. 13, 2025 and entitled “SYSTEM FOR REPRESENTING A QUALITY AND HEALTH OF AN INDIVIDUAL MICROBIOME,” which is incorporated herein by reference in its entirety.

### BACKGROUND

Today, individuals have little insight into their personal microbiomes even though the composition, quality, and health of an individual's microbiome may have a large impact on their long term health. In some cases, if an individual is aware that they have a weak microbiome, the individual may be able to take proactive action to improve the microbiome. Some companies offer services for improving health of an individual's microbiome via pre-planned meals or pre-planned recipes as a controlled nutritional input to the individual. Typically, these services provide at home meal delivery or weekly notifications of recipes and associated shopping lists. Unfortunately, these services provide no rapid insight to the effect on the individual's microbiome, and, as a result, often result in the individual's participation waning over time and, therefore, not achieving their microbiome improvement goals.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is an example block diagram of a system for generating and maintaining an avatar representing a health or status of an individual's microbiome, according to some implementations.

FIG. 2 is a flow diagram illustrating an example process associated with the system for providing personalized microbiome avatar as discussed above with respect to FIG. 1, according to some implementations.

FIG. 3 is a flow diagram illustrating an example process associated with the system for updating a status of an avatar, according to some implementations.

FIG. 4 is a flow diagram illustrating an example process associated with the system for recommending meals or snacks to assist in the improvement of the avatar, according to some implementations.

FIG. 5 is an example system for providing a microbiome avatar or visual representation according to some implementations.

FIG. 6 is an example user device that may implement the techniques described herein according to some implementations.

FIG. 7 is an example pictorial diagram illustrating an example user interface associated with a microbiome avatar according to some implementations.

FIG. 8 is an example pictorial diagram illustrating an example user interface associated with a microbiome avatar according to some implementations.

FIG. 9 is an example pictorial diagram illustrating an example user interface associated with a microbiome avatar according to some implementations.

FIG. 10 is an example pictorial diagram illustrating an example user interface associated with a microbiome avatar according to some implementations.

FIG. 11 is a series of example pictorial diagrams illustrating example user interfaces associated with a microbiome avatar according to some implementations.

FIG. 12 is an example pictorial diagram illustrating an example user interface 1200 associated with capturing consumption data for use in maintaining a microbiome avatar according to some implementations.

FIG. 13 is an example pictorial diagram illustrating an example user interface associated with an output or response to capturing consumption data for use in maintaining a microbiome avatar according to some implementations.

FIG. 14 is an example pictorial diagram illustrating an example user interface associated with a microbiome avatar after a meal is logged according to some implementations.

FIG. 15 is an example pictorial diagram illustrating an example user interface associated with a microbiome avatar according to some implementations.

FIG. 16 is an example pictorial diagram illustrating an example user interface associated with a microbiome avatar according to some implementations.

FIG. 17 is a series of example pictorial diagrams illustrating example user interfaces associated with a microbiome avatar according to some implementations.

FIG. 18 is a series of example pictorial diagrams illustrating example user interfaces associated with a microbiome avatar according to some implementations.

FIG. 19 is an example pictorial diagram illustrating an example user interface associated with a microbiome avatar according to some implementations.

FIG. 20 is an example pictorial diagram illustrating an example user interface associated with a microbiome avatar according to some implementations.

FIG. 21 is an example pictorial diagram illustrating an example user interface associated with a microbiome avatar according to some implementations.

FIG. 22 is a series of example pictorial diagrams illustrating an example a microbiome avatar with various accessories according to some implementations.

### DETAILED DESCRIPTION

Discussed herein are systems, applications, and user interfaces for providing personalized microbiome and/or gut health monitoring and, in some cases, meal planning. In various implementations, the system discussed herein may include a cloud-based service and an application hosted on a user device for receiving user input, such as data associated with meals, snacks, or other nutrition, and provide recommendations, such as providing meal plans, health-based and nutrition-based recommendations that, in some cases, may be personalized based at least in part on the biology (e.g., the microbiome of a participant's digestive system, blood sugar and blood fat postprandial responses, fasted blood tests, age, allergies, physical location, overall health and wellness, other demographic data, and the like) of each participant.

As discussed herein, a microbiome refers to the collection of microbes that inhabit a human body. These microbes may include, but are not limited to, bacteria, fungi, viruses, as well as other microscopic life forms. The human microbiome may include trillions of microbes or microbes that live in and on an individual's digestive system. In a microbiome, there are various types of microbes, some of which may be considered good species, beneficial microbes, or pro-health microbes, while others can be considered bad species or harmful microbes. The beneficial microbes provide benefits to their host individual including maintaining human health, aiding in digestion, producing vitamins, preventing inflammation and autoimmune reactions, training the immune system to recognize and fight off harmful pathogens, and the like. However, harmful microbes within a microbiome may cause infections, disease, otherwise disrupt the beneficial microbes (which can contribute to the development of various health conditions), produce toxins, and the like.

In some cases, the system may represent the microbiome of the individual as an avatar (or virtual character) that illustrates attributes of their personal microbiome health based on the individual's actual nutritional consumption. For example, the avatar may appear healthy, happy, agreeable, friendly, well, and the like when the associated individual is consuming food that encourages growth of a healthy microbiome, while the avatar may appear unhealthy, sad, irritable, weak, and the like when the associated individual is consuming food that is harmful or disruptive to a healthy microbiome. In other cases, the avatar's colors may change, shine or luster may increase or decrease, size may increase or decrease, position relative to the display (e.g., further in the background or present in the foreground), awakeness level, energy level, and the like may vary based on the food that the associated individual is consuming. In this manner, the individual may visibly recognize how other nutritional consumption directly affects their microbiome as the avatar undergoes personal, situational and lifecycle style changes.

In some situations, the environment surrounding the avatar and/or accessories associated with the avatar may be updated. For example, articles of clothing worn by the avatar, a piece of equipment associated with the avatar, an activity performed by the avatar, or an venue/locations (e.g., the current virtual environment) around the avatar, and/or the like may be updated. For example, after the individual consumes a healthy meal the avatar may be updated to include an outfit or other accessory that shows that the avatar and the individual's microbiome has had a positive experience (e.g., the individual's microbiome is benefiting from the meal). For instance, the avatar may be updated to include sporting accessories (e.g., a jersey, a racket, vizor, ball, cleats, dumbbells, and/or the like) to illustrate to the user that the avatar is energized, healthy, and ready to go. In some cases, the avatar may be transported to a gym and wear a headband and/or weightlifting gloves to show that now that the individual has consumed a healthy meal, the avatar is ready to exercise. In this manner, the avatar may both act to inform the user that the microbiome is healthy (as a response to the individual's healthy eating) and encourage the individual to take other health conscious steps (e.g., promoting exercise).

In some cases, as the user regularly consumes healthy food or food that is beneficial to their microbiome, the avatar associated with the individual may undergo more permanent changes, such as evolutions, lifecycle stage changes (e.g., changes in age, change from infant to toddler, adolescent to adult, and/or the like), multiplication or duplication (e.g., the number of avatars may increase), and/or the like. For instance, in some cases, the avatar may level up and/or down based on the nutritional consumption of the associated individual over one or more periods of time (e.g., a day, a week, a month, a year, and/or the like). In some specific cases, the avatar may also regress or level down in response to unhealthy or disruptive consumption habits of the associated user. For example, if the user regularly consumes unhealthy highly processed foods that are known to disrupt the operations of a healthy microbiome, the avatar may regress backwards.

In the various examples, the changes to the avatar associated with the individual are a direct result of the consumption habits of the associated individual. In these cases, the individual may capture and/or input data associated with their consumption into the user device on an ongoing basis to track their consumption habits, nutritional intake, quality of food consumed, and the like. For example, the system may include an application hosted on a user device that may enable a participant to capture food data associated with meals (e.g., photos or image data of meals, snacks, supplements, and the like prior to consumption), grocery shopping receipts, contents of a food storage area (e.g., a pantry, refrigerator, cold storage, freezer, or the like), and to provide the associated captured data to the cloud-based system. As one specific example, the individual may utilize the user device to capture image data of each item the individual is preparing to consume. In this manner, the system may have image data representing the items consumed by the individual and a time stamp (e.g., the time stamp associated with the image capture time) for each item consumed. The captured data including any image data may then be provided to a cloud-based system and/or processed in full or in part on the user device.

In some cases, the avatar may adjust its hair style, eye color, fur style, and/or other temporary appearance attribute or characteristic in response to the nutritional data determined by the system or other user inputs, such as a user selection. In this manner, the avatar and the environment surrounding the avatar may undergo changes that are situational (or temporary), such as a trip to the gym while also undergoing more permanent changes, such as when the avatar levels up from continuous positive eating or the system detects a habit change or pattern of positive eating.

In some cases, the cloud-based system may include one or more machine learned models that are trained to segment, classify, detect foods and ingredients, from image data and the like and to output or generate nutritional data associated with each detected food or ingredient. For example, the one or more machine learned models may be configured to disambiguate between different items, foods, or ingredients within image data of a meal, plate, and/or bowl of food, and/or the like. The one or more machine learned models may classify each of the detected items, such as classifying items between types of foods, such as meats and vegetables, between types of each class (e.g., chicken from pork or the like) as well as between base ingredients of types such as whole grain bread, white bread, wheat bread, or the like. In some cases, the system and/or the one or more machine learned models may determine attributes or characteristics of the classified ingredients. For example, the one or more machine learned models may output size of the meal, and plant diversity, protein diversity, weight of the ingredient, number of ingredients, and/or the like. In some cases, the class of the ingredient may be associated with particular nutritional data such as based on weight, quantity, volume, amount, and/or the like.

In some cases, the one or more machine learned models may also determine nutritional data associated with each classified item. For example, the system may determine a calorie count, fiber content, fat quantity and quality (e.g., monounsaturated or polyunsaturated, saturated, and the like), carbohydrate quantity and quality (e.g., glycemic index), vitamin content, mineral content, protein type (e.g., plant-based or animal-based) and content, salt/sodium content, water content, polyphenol content, level of processing (e.g., NOVA) and the like. In some examples, the one or more machine learned models for segmenting, classifying, detecting foods and ingredients, and generating data associated with each detected food or ingredient may be trained on image data with associated food data as well as known nutritional data.

The machine learned models and/or network (such as additional machine learning models) may also be trained using third party health data and microbiome data to generate weights or factors for items consumed based on individual species or microbes that could potentially be present in an individual's personal microbiome. In some cases, the weights may be positive, negative, or neutral depending on determined beneficial and/or harmful tendencies. These weights may then be used to generate microbiome metrics for the items consumed based on the expected effect to the individual's microbiome (e.g., the expected effect on the abundances of different species of microbes). The microbiome metrics for the items consumed may represent an expected effect to the overall quality of an individual's overall microbiome. For example, Lawsonibacter asaccharolyticus growth is associated with coffee consumption and, accordingly, consumption of coffee may provide an expected positive affected on the individual's microbiome. Therefore, coffee may have a positive microbiome metric associated therewith.

In some examples, the system may also receive participant data associated with an individual to further personalize the avatar's appearance, health, and wellbeing. The participant data may include microbiome data, such as microbiome data identifying the presence and/or quantity of various bacteria and the like. For example, the participant data may include lab generated data, such as when a participant provides biological samples to a lab for testing. For instance, a participant may provide a lab with a sample, such as a stool sample, for microbiome analysis. As an example, metagenomic testing can be performed using the sample to allow the DNA of a microbiome of an individual to be digitalized. Generally, a microbiome analysis includes determining the composition and/or function of a community of microorganisms in a particular location, such as within the gut of a user. An individual's microbiome appears to have a strong causal relationship to metabolism, weight and health, yet only ten to thirty percent of the microbiome is common across different individuals.

The participant data may also include health data, blood data, glucose data, ketone data, nutrition data, genetic data, saliva data, biometric data, questionnaire data, diet data (e.g., food frequency questionnaire), psychological data (e.g., hunger, sleep quality, mood, and the like), objective health data (e.g., age, sex, height, weight, medical history, and the like) as well as other types of data. Generally, health data may refer to any psychological, subjective and/or objective data that relates to and is associated with one or more individuals. The health data might be obtained through testing, self-reporting (such as via the application hosted on the user device), and the like.

In some examples, the health data includes wearable data obtained from technology worn and/or utilized by a participant. For instance, a participant may wear a fitness device, such as an activity-monitoring device, that monitors motion, heart rate, determines how much a participant has slept, the number of calories burned, activities performed, blood pressure, body temperature, and the like. The participant may also wear a continuous glucose meter that monitors blood glucose levels often by measuring levels of glucose in interstitial fluid.

A participant may also provide data that may be utilized to predict the target values and/or changes to the target values and generate the nutritional recommendations using other devices such as blood glucose monitors, finger pricks which in some examples are used with dried blood spot cards, blood pressure monitors, and the like. A participant may also input data into one or more software applications (or provide the data some other way) that may be utilized. For example, a participant may enter the demographic information (e.g., age, sex, cultural heritage, and/or the like), how much the participant slept, what exercise the participant performed during a given period of time, how hungry the participant is at one or more times of day including mealtimes, how the participant feel, what medication the participant consumes, and the like. As another example, a participant or a lab may provide test data determined from one or more tests, such as urinalysis test strips, blood test strips, and the like. The test data may come from different sources, such as but not limited to from one or more of an individual, a lab, a doctor, an organization, and/or some other data source. A participant may also provide data about their food preferences, medical guidance the participant has received, or personalized food constraints/preferences, such as allergies, being vegan, gluten free, KETO or other adhered diet, kosher, halal, or the like.

In some implementations, utilizing the health data personalized for an individual, the nutritional data, and/or the microbiome metrics, the system may generate (e.g., via one or more machine learned models, algorithmic techniques, heuristics, or the like) personalized food scores or metrics for each food, such as a paring of the individual to the food or the food to a general population. For example, baby kale for a first individual may have a first score while for a second individual the same baby kale may have a second score different than the first score. In this manner, each individual may have a personalized food score for each item (e.g., food, ingredient, supplement, product, and the like). The system may also generate (e.g., via one or more machine learned models, algorithmic techniques, heuristics, or the like) utilizing the health data a personalized list of gut boosters (e.g., positive items for the participants microbiome) and gut suppressors (negative items for the participants microbiome). In some cases, the system may also generate (e.g., via one or more machine learned models, algorithmic techniques, heuristics, or the like) in addition to or in lieu of participant specified preferences, dietary preferences and/or exclusions.

In the various examples, the system may translate or otherwise utilize the health data, nutritional data, microbiome metrics, and/or any personalization food scores for the individual to cause visible changes to the avatar associated with the individual. For example, the system may input the health data, nutritional data, microbiome metrics, and/or any personalization food scores as well as the individual's current avatar into one or more additional machine learning models that may be trained to output an adjusted or modified avatar. In this manner, the avatar may change based on the items consumed by the individual and the nutritional value of those items.

In some cases, the avatar may be associated with one or more thresholds or metrics that when meet or exceeded by the individual may cause the avatar to undergo changes (such as lifecycle transitions, evolutions, duplications or multiplications, and/or the like). In some cases, these thresholds may be set by the individual, set by the system, determined by the system based at least in part on personalization data associated with the individual (e.g., via one or more machine learned models or networks), and/or the like. For example, the system may set the thresholds for an individual based on the individual demographic data and health data entered via the user device during initialization phase of the avatar. In some cases, the number of thresholds for an individual may vary based on the circumstances associated with the individual or be uniform across multiple individuals (e.g., systemwide settings thresholds).

In some examples, the avatar associated with an individual may be configured to perform various actions in response to detected consumption data by the individual. For instance, the avatar may be configured to perform a celebratory action in response to the individual consuming a meal having more than a threshold level of nutritional value or pro microbiome aspects. As an illustrative example, the user may capture image data of a meal high in fiber, plant diversity, fat content and quality, and/or the like. The system may determine a level of fiber, plant diversity, fat content and quality, and/or the like by processing the image data (e.g., via one or more machine learned models or networks) and comparing the determined levels to one or more meal-based thresholds. In this example, when one or more of the one or more meal-based thresholds are met and/or exceeded, the system may cause the avatar to perform a celebratory action.

In some specific examples, such as when the individual is part of a group, the avatar associated with the individual may be configured to interact with other avatars associated with other members of the group. For example, when the avatar associated with the individual is to perform a celebratory action the avatar may be configured to visit or otherwise interact with other group member's avatars to provide a community celebration and, thereby, provide community reinforcement to the healthy eating habits of the individual and the other group members. In some cases, the other group members may via the system provide feedback and/or congratulations to the individual in response to viewing the celebratory action or event of the group of avatars.

In some implementations, the individual may personalize their avatar during initialization stage or process. For example, the system may provide a list or menu of various visual representations of avatars corresponding to various types of real life microbes often found in human microbiomes. In some cases, the individual may also select color, eye color, eye style, hair or fur style, clothing, equipment, base size, and/or other appearance personalization of the avatar. In some cases, the individual may create their avatar using a character creation interface such as those known in the art having various sliders and personalization selections.

In one specific implementation, such as when the user undergoes and microbiome test, such as a lab based test, the system may select or initialize the appearance of the avatar associated with the individual based at least in part on one or more detected microbes within the individual's microbiome. For example, the system may select the initial appearance based on a look and feel of a microbe having high quantity or presence within the individual's microbiome and/or having known positive health implications. In these examples, the individual may still customize the initialized avatar as discussed above.

In some cases, the system may also generate one or more meals or recipes for the individual based at least in part on the current state of the individual's microbiome to further progress the user avatar, such as to evolve, grow, or otherwise improve the health of the individual's microbiome and corresponding avatar. For example, the system may attempt to balance the meal planning based on the individuals taste preferences and consumption habits. In some instances, the system may present multiple alternative recipes or meal plan options based on an item list provided by the individual (e.g., via the user device) to allow the participant a choice of meals during the time period.

In some examples, the meal planning and recipes may be the output of one or more machine learned models that receive the time period food data and the health data for each participant in the group. For example, the one or more recipes and meal planning machine learned models may be trained on food data and heath data for individual participants and/or groups with various numbers of participants.

As described herein, various machine learned models or sets of models may be utilized by the system. In various examples, the sets of machine learned models may be the same or part of the same set or different sets to produce different results or outputs. The machine learned models may be generated using various machine learning techniques. For example, the models may be generated using one or more neural network(s). A neural network may be a biologically inspired algorithm or technique which passes input data (e.g., image and sensor data captured by the IoT computing devices) through a series of connected layers to produce an output or learned inference. Each layer in a neural network can also comprise another neural network or can comprise any number of layers (whether convolutional or not). As can be understood in the context of this disclosure, a neural network can utilize machine learning, which can refer to a broad class of such techniques in which an output is generated based on learned parameters.

As an illustrative example, one or more neural network(s) may generate any number of learned inferences or heads from the captured sensor and/or image data. In some cases, the neural network may be a trained network architecture that is end-to-end. In one example, the machine learned models may include segmenting and/or classifying extracted deep convolutional features of the sensor and/or image data into semantic data. In some cases, appropriate truth outputs of the model in the form of semantic per-pixel classifications (e.g., vehicle identifier, container identifier, driver identifier, and the like).

Although discussed in the context of neural networks, any type of machine learning can be used consistent with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3 ), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like. In some cases, the system may also apply Gaussian blurs, Bayes Functions, color analyzing or processing techniques and/or a combination thereof.

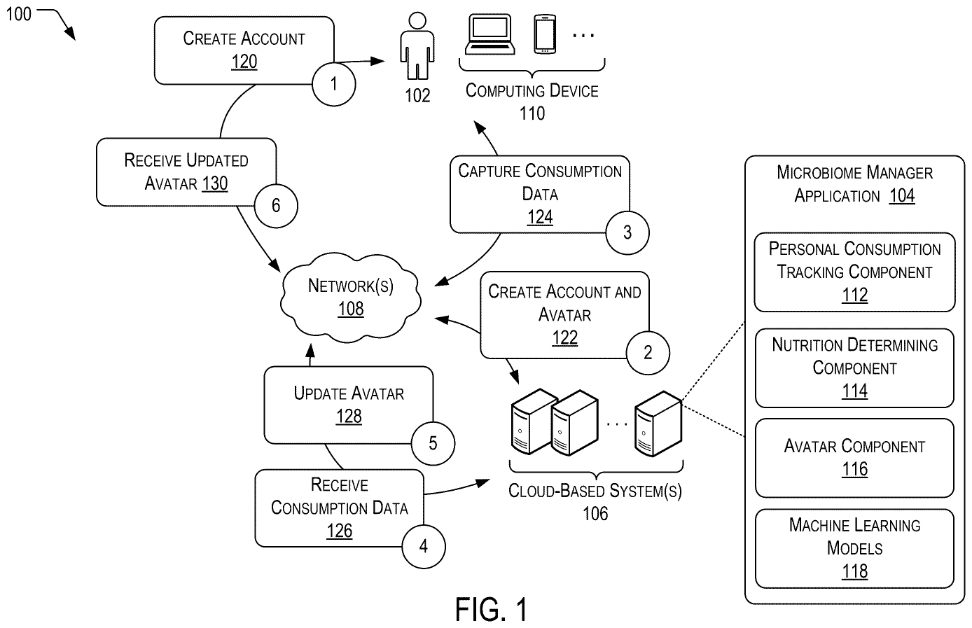

FIG. 1 is a view of an example system 100 usable to provide a visual representation of an individual's microbiome, such as via a growing microbe avatar according to some implementations. In some examples, the system 100 may include a user 102 that may interact with a microbiome manager application 104 hosted on a cloud-based system 106 via a network 108 using computing devices, generally indicated by 110.

In the illustrated example, the microbiome manager application 104 may include a personal consumption tracking component 112, a nutrition determining component 114, an avatar component 116, and one or more machine learning models 118. The personal consumption tracking component 112 may be configured to receive consumption data from the user 102 via the computing device 110. For example, the consumption data may include image data and/or sensor data of a meal or snack that the user 102 to is preparing to consume, user input data associated with the ingredients of the meal or snack the user 102 is preparing to consume, audio data describing the meal or snack that the user 102 is preparing to consume, and/or the like. In some cases, the personal consumption tracking component 112 may log the consumption data together with a timestamp associated with the time of consumption by the user 102. The personal consumption tracking component 112 may also log the ingredients, nutritional data, and/or the like associated with the consumption data.

The nutritional determining component 114 may be configured to process the consumption data received from the user 102 via the computing device 110, such as to determine ingredients, nutritional data associated with the ingredients, and/or other attributes or characteristics of the meal or snack being consumed by the user 102. In some cases, the personal consumption tracking component 112 may log or record the nutritional data, ingredients, and/or the attributes and characteristics determined by the nutritional determining component 114. In some cases, the nutritional determining component 114 may utilize the one or more machine learning models 118 to determine ingredients, nutritional data associated with the ingredients, and/or other attributes or characteristics of the meal or snack being consumed by the user 102. For instance, the one or more machine learning models 118 may be configured to segment image data associated with a meal, such as a photograph captured by the user 102 using the computing device 110. The one or more machine learning models 118 may then classify each item or object within the segmented image data to determine the type or identity of the ingredients, quantity or volume of each ingredient, and/or the like. The one or more machine learning models 118 may utilize the classified ingredients as well as the quantity or volume to determine nutritional data associated with the meal represented in the image data provided by the user 102.

The avatar component 116 may be configured to visually represent a status (e.g., health, happiness, quality, quantity, presence, and/or the like) of one or more microbes present in the microbiome of the user 102. In some cases, the avatar component 116 may be configured to allow the user 102 to customize and/or select an initial avatar representing the microbiome of the user 102, such as via one or more character or avatar creation tools. The avatar component 116 may also be configured to receive the consumption data and/or nutritional data determined by the nutritional determining component 114 and to cause the status of the avatar or visual representation to change based at least in part on the consumption data and/or nutritional. For example, as the user 102 consumes items having a known positive effect on a microbiome the avatar component 116 may cause a positive change (e.g., happy appearance, lifecycle progression, increased energy levels, awakeness, and/or the like) to the status of the avatar visible to the user 102. Similarly, as the user 102 consumes items having a known negative effect on a microbiome, the avatar component 116 may cause a negative change (e.g., a sad or irritated mood, regression and lifecycle, decreased energy levels, sleep or lethargy, and/or the like) to the status of the avatar visible to the user 102.

With respect to FIG. 1, the user 102 may be creating an account or avatar. For instance, in the illustrated example, the user 102 may, at operation 120 (indicated by “1”), create a new account or avatar. For instance, the user 102 may create an account in a manner of an individual user creating a personal account with various types of known downloadable applications or social sharing sites. In some cases, the user 102 may specify identifying content (e.g., account name, address, and the like), background content (e.g., purpose, goals, diet, and the like), personal data (e.g., health data, demographic data, cultural data, lifestyle/exercise data, and/or the like), as well as an initial appearance of the microbiome avatar, as discussed herein.

In the illustrated example, at operation 122, (indicated by “2”), the microbiome manager application 104 may create the account and the associated avatar. For example, the microbiome manager application 104 may allow the user 102 to customize the avatar, such as via character creation tools, one or more sliders, one or more selection tools, and/or the like. In some specific implementations, the user 102 may provide, as part of the account creation process, a lab test or other data representing their personal microbiome. In these specific implementations, at operation 122, the microbiome manager application 104 may generate an initial avatar based on the personal microbiome data provided by the user 102. In this manner, the avatar may represent the actual microbiome of the user 102. For instance, the microbiome manager application 104 may select an avatar similar to in appearance to a microbe having a high level of presence in the microbiome of the user 102. As another instance, the microbiome manager application 104 may select the avatar based on a rarity of the microbe, the specific microbe having positive affect on the long-term health of the user 102, the specific microbe that the microbiome manager application 104 desires to encourage growth of, and/or the like.

Next, at operation 124 (indicated by “3”), the user 102 may, via an application hosted on the computing device 110, capture consumption data associated with a meal or snack (e.g., solid or liquid) the user 102 is preparing to consume. As discussed above, the user 102 may capture image data of the meal or snack via the computing device 110. As another example, the user may select the ingredients and/or quantity via a drop-down list provided by the application hosted on the computing device 110 or the user 102 may enter the ingredients and/or quantity a user input interface.

At operation 126 (indicated by “4”), the Microbiome manager application 104 may receive the consumption data (e.g., the image data representing the meal or snack) from the user 102. Next at operation 128 (indicated by “5”), the Microbiome manager application 104 may update the status of the avatar or visual representations of the microbiome of the user 102. For instance, as discussed above, the nutritional determining component 114 may determine the ingredients and/or nutritional data associated with consumption data and the avatar component one 116 may update the status of the avatar based at least in part on the nutritional data.

In this example, at operation 130 (indicated by “6”), the user 102 may view the updated avatar on a display of the computing device 110. In this example, as the appearance or status of the avatar is directly related to the health and/or effect of the consumption data on the microbiome of the user 102, the user 102 may easily infer the health and status of their actual microbiome and/or the long-term effects of the user's eating habits.

In the illustrated example, each of the computing devices 110 may include one or more processors and memory storing computer executable instructions to implement the functionality discussed herein attributable to the various computing devices. In some examples, the computing device 110 may include desktop computers, laptop computers, tablet computers, mobile devices (e.g., smart phones or other cellular or mobile phones, mobile gaming devices, portable media devices, etc.), or other suitable computing devices. The computing device 110 may execute one or more client applications, such as a web browser (e.g., Microsoft Windows Internet Explorer, Mozilla Firefox, Apple Safari, Google Chrome, Opera, etc.) or a native or special-purpose client application (e.g., hosted applications, messaging applications, email applications, games, etc.), to access and view content over the network 108.

The network 108 may represent a network or collection of networks (such as the Internet, a corporate intranet, a virtual private network (VPN), a local area network (LAN), a wireless local area network (WLAN), a cellular network, a wide area network (WAN), a metropolitan area network (MAN), or a combination of two or more such networks) over which the computing device 110 may access the system 106 and/or communicate with one another.

The system 106 may include one or more servers or other computing devices, any or all of which may include one or more processors and memory storing computer executable instructions to implement the functionality discussed herein attributable to the social networking system or digital platform. The system 106 may enable the user 102 to interact with the system 106 and with other users.

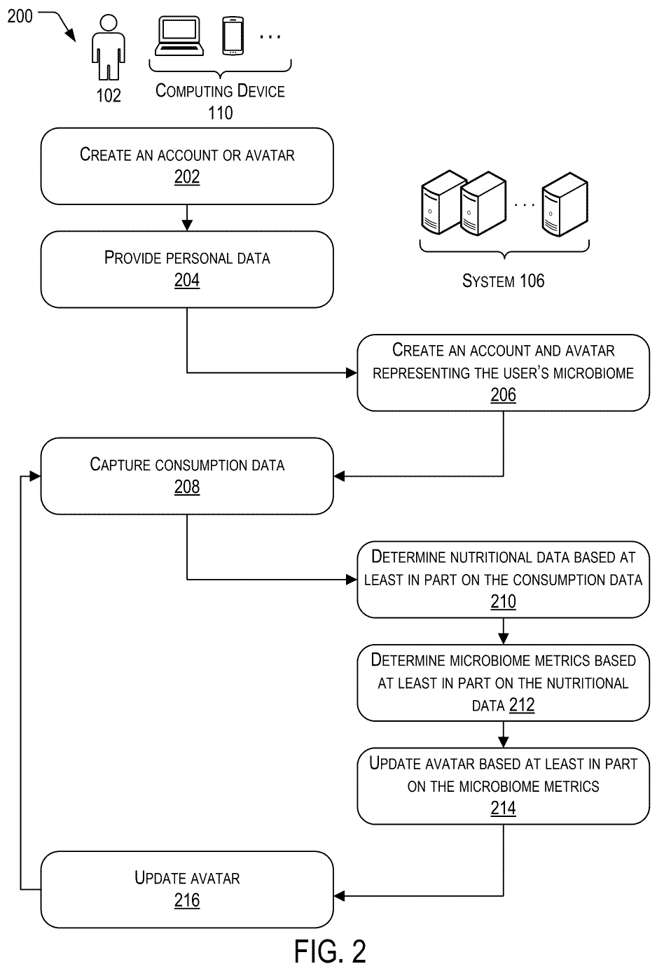

FIGS. 2-4 are flow diagrams illustrating example processes associated with the system discussed herein. The processes are illustrated as a collection of blocks in a logical flow diagram, which represent a sequence of operations, some or all of which can be implemented in hardware, software, or a combination thereof. In the context of software, the blocks represent computer-executable instructions stored on one or more computer-readable media that, when executed by one or more processor(s), perform the recited operations. Generally, computer-executable instructions include routines, programs, objects, components, encryption, deciphering, compressing, recording, data structures and the like that perform particular functions or implement particular abstract data types.

The order in which the operations are described should not be construed as a limitation. Any number of the described blocks can be combined in any order and/or in parallel to implement the processes, or alternative processes, and not all of the blocks need be executed. For discussion purposes, the processes herein are described with reference to the frameworks, architectures and environments described in the examples herein, although the processes may be implemented in a wide variety of other frameworks, architectures or environments.

FIG. 2 is a flow diagram illustrating an example process 200 associated with the system 106 for providing personalized microbiome avatar as discussed above with respect to FIG. 1, according to some implementations. As discussed herein, a system, such as a cloud-based system in communication with one or more users 102 via the user devices 110 to generate and update an avatar or visual representation of the microbiome of the user 102 over time.

At 202, the user 102 may create a new account or avatar and, at 204, the user 102 may provide personal data to assist with the account creation and/or avatar creation. For instance, the user 102 may create an account in a manner of an individual user creating a personal account with various types of known downloadable applications or social sharing sites. In some cases, the user 102 may specify identifying content (e.g., account name, address, and the like), background content (e.g., purpose, goals, diet, and the like), personal data (e.g., health data, demographic data, cultural data, lifestyle/exercise data, and/or the like), as well as an initial appearance of the microbiome avatar, as discussed herein. For example, the user 102 may customize the avatar, such as via character creation tools, one or more sliders, one or more selection tools, and/or the like. In some specific implementations, the user 102 may provide as part of the account creation process a lab test or other data representing their personal microbiome.

At 206, the system 106 may create the account and the associated avatar. For instance, the system 106 may generate an initial avatar based on the personal microbiome data provided by the user 102. In this manner, the avatar may represent the actual microbiome of the user 102. In other cases, such as when personal lab tests are provided, the system 106 may selected an avatar similar to in appearance to a microbe having a high level of presence in the microbiome of the user 102. As another instance, the microbiome manager application 104 may select the avatar based on a rarity of the microbe, the specific microbe having positive affect on the long-term health of the user 102, the specific microbe that the microbiome manager application 104 desires to encourage growth of, and/or the like.

At 208, the user 102 may capture, via an application hosted on the computing device 110, consumption data associated with a meal or snack (e.g., solid or liquid) the user 102 is preparing to consume. As discussed above, the user 102 may capture image data of the meal or snack via the computing device 110. As another example, the user may select the ingredients and/or quantity via a drop-down list provided by the application hosted on the computing device 110 or the user 102 may enter the ingredients and/or quantity in a user input interface.

At 210, the system 106 may receive the consumption data (e.g., the image data representing the meal or snack) from the user 102 and determine nutritional data based at least in part on the consumption data. In some cases, the system 106 may be configured to process the consumption data received from the user 102 to determine ingredients, nutritional data associated with the ingredients, and/or other attributes or characteristics of the meal or snack being consumed by the user 102.

In some cases, the system 106 may utilize the one or more machine learning models to determine ingredients, nutritional data associated with the ingredients, and/or other attributes or characteristics of the meal or snack being consumed by the user 102. For instance, the one or more machine learning models may be configured to segment image data associated with a meal, such as a photograph captured by the user 102 using the computing device 110. The one or more machine learning models may then classify each item or object within the segmented image data to determine the type or identity of the ingredients, quantity or volume of each ingredient, and/or the like. The one or more machine learning models may utilize the classified ingredients as well as the quantity or volume to determine nutritional data associated with the meal represented in the image data provided by the user 102.

At 212, the system 106 may determine one or more microbiome metrics based at least in part on the nutritional data. For example, the system 106 may determine an effect of the nutritional data to one or more microbes either known to be present in a microbiome of the user 102 or commonly present in the microbiomes of individuals. For example, the system 106 may apply a heuristic, algorithmic, and/or other approach to determining the effect of the nutritional data on each microbe and/or the microbiome of the user 102 and/or the microbiome of a typical individual. In some specific examples, the system 106 may utilize one or more machine learned models trained to receive nutritional data as input and to output the microbiome metrics.

At 214, the system may update the status/appearance of the avatar or visual representations of the microbiome of the user 102 based at least in part on the microbiome metrics and, at 216, the computing device 110 may present the updated avatar on the display. For instance, as discussed above, the ingredients, quantities or amounts of each ingredient, together with a known effect of each ingredient on an individual's microbiome may be utilized to determine an expected positive or negative transformation of the microbes in the microbiome of the user 102 as represented by the microbiome metrics. The positive or negative transformation may be translated by the system 106 into a visible change to the status of the avatar that may be viewed by the user 102 on a display of the computing device 110.

The process 200 may then return to 208 as the user 102 prepares to consume their next meal or snack. In some cases, the microbiome metrics may be cumulative, associated with periods of time (such as a sliding window type approach), and/or the like. In this manner, the microbiome metrics may be associated with an ingredient and/or represent the expected change of in the microbiome of the user 102 over the period of time or as an overall long term health. For instance, one of the metrics may represent an expected change in the quantity of a particular microbe in the microbiome of the user 102. As the user 102, consumes items that boots growth of this microbe, the microbiome metric may increase over time (such as an addition or summation of growth). However, as the user 102 fails to consume items that promote the growth of the specific microbe, the associated microbiome metric may decline (such as to represent an expected decrease in a quantity of the microbe).

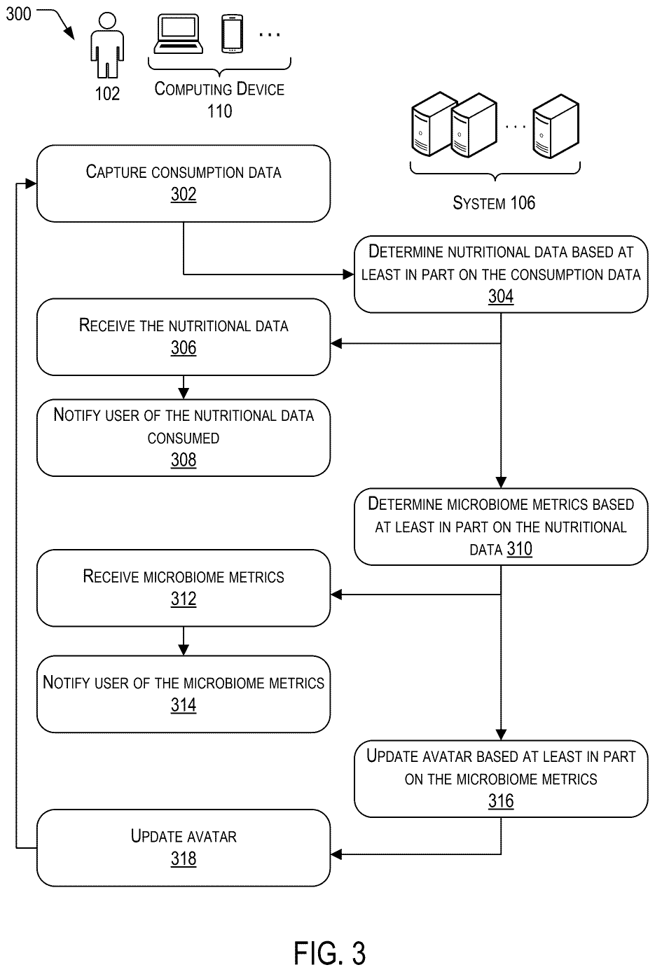

FIG. 3 is a flow diagram illustrating an example process 300 associated with the system 102 for updating a status of an avatar, according to some implementations. As discussed herein, a user 102 may utilize an application hosted on a computing device 110 operating in conjunction with a cloud-based system 106 to maintain an avatar of visual representation of the microbiome of the user 102 over time that the user 102 may utilize to infer an overall status or health of their microbiome.

At 302, the user 102 may, via an application hosted on the computing device 110, capture consumption data associated with a meal or snack (e.g., solid or liquid) the user 102 is preparing to consume. As discussed above, the user 102 may capture image data of the meal or snack via the computing device 110. As another example, the user may select the ingredients and/or quantity via a drop-down list provided by the application hosted on the computing device 110 or the user 102 may enter the ingredients and/or quantity a user input interface.

At 304, the system 106 may receive the consumption data (e.g., the image data representing the meal or snack) from the user 102 and determine nutritional data based at least in part on the consumption data. In some cases, the system 106 may be configured to process the consumption data received from the user 102 to determine ingredients, nutritional data associated with the ingredients, and/or other attributes or characteristics of the meal or snack being consumed by the user 102.

In some cases, the system 106 may utilize the one or more machine learning models to determine ingredients, nutritional data associated with the ingredients, and/or other attributes or characteristics of the meal or snack being consumed by the user 102. For instance, the one or more machine learning models may be configured to segment image data associated with a meal, such as a photograph captured by the user 102 using the computing device 110. The one or more machine learning models may then classify each item or object within the segmented image data to determine the type or identity of the ingredients, quantity or volume of each ingredient, and/or the like. The one or more machine learning models may utilize the classified ingredients as well as the quantity or volume to determine nutritional data associated with the meal represented in the image data provided by the user 102.

At 306, the computing device 110 may receive the nutritional data and, at 308, the user 102 may be notified of the nutritional data consumed. For example, the nutritional data may be logged by the system 106 and provided to the device 110. The computing device 110 may determine one or more particular elements of the nutritional data is noteworthy. For example, a quantity of the particular element may meet or exceed one or more thresholds (e.g., their element specific thresholds or overall thresholds). For instance, if the user consumed more than a threshold amount of fiber, the hosted applications may cause a notification or alert to be triggered on the computing device 110 to inform the user 102 of the healthy eating.

At 310, the system 106 may determine one or more microbiome metrics based at least in part on the nutritional data. For example, the system 106 may determine an effect of the nutritional data to one or more microbes either known to be present in a microbiome of the user 102 or commonly present in the microbiomes of individuals. For example, the system 106 may apply a heuristic, algorithmic, and/or other approach to determining the effect of the nutritional data on each microbe and/or the microbiome of the user 102 and/or the microbiome of a typical individual. In some specific examples, the system 106 may utilize one or more machine learned models trained to receive nutritional data as input and to output the microbiome metrics.

At 312, the computing device 110 may receive the microbiome metrics and, at 314, the user 102 may be notified of the microbiome metrics. For example, the microbiome metrics may be logged by the system 106 and provided to the device 110. The computing device 110 may determine one or more particular microbes of the microbiome metrics is expected to have undergone a positive effect. For example, if an ingredient that supports the growth of the particular microbe is consumed in quantity then the user 102 may be notified of the expected change to the microbe (e.g., that the microbe has multiplied). In some cases, the notifications may inform the user 102 of the positive effects that the particular microbe has a long term health of the user 102.

At 316, the system may update the status/appearance of the avatar or visual representations of the microbiome of the user 102 based at least in part on the microbiome metrics. For instance, as discussed above, the ingredients, quantities or amounts of each ingredient, together with a known effect of each ingredient on an individual's microbiome may be utilized to determine an expected positive or negative transformation of the microbes in the microbiome of the user 102 as represented by the microbiome metrics. The positive or negative transformation may be translated by the system 106 into a visible change to the status of the avatar that may be viewed by the user 102 on a display of the computing device 110.

At 318, the updated avatar may be presented on the display of the computing device 110, such that the user may infer the effect of their eating on their long term health and microbiome. In some examples, the updated status/appearance of the avatar may include a change in lifecycle, such as a transition from adolescence to adulthood, a leveling up, such as a change in size, color, physique, and/or the like, a change in health, such as appearing tired, sick, energetic, and/or the like, or a change in mood or personality, such as happiness, irritation, sadness, and/or the like. In some examples, the situation in which the avatar is presented may be updated, such as an article of clothing worn by the avatar, a piece of equipment associated with the avatar, an activity performed by the avatar, or an environment around the avatar. In some cases, a number or quantity of avatars may change. For example, the avatar may appear as a community of multiple avatars wherein each avatar represents one or more specific microbes within the microbiome of the user 102. In this manner, an increase in the number of avatars may indicate an overall healthy microbiome, while a decrease in the number of avatars may indicate an overall unhealthy microbiome. In some cases, the avatar may appear as a community of multiple avatars wherein some avatars represent beneficial microbes which are positive for human health, while other avatars represent harmful microbes which are detrimental for human health.

The process 300 may then return to 302 as the user 102 prepares to consume their next meal or snack. In some cases, the microbiome metrics may be cumulative, associated with periods of time (such as a sliding window type approach), and/or the like. in this manner, the microbiome metrics may represent the expected change of in the microbiome of the user 102 over the period of time or as an overall long term health. For instance, one of the metrics may represent an expected change in the quantity of a particular microbe in the microbiome of the user 102. As the user 102, consumes items that boosts the growth of this microbe, the microbiome metric may increase over time (such as an addition or summation of growth). However, as the user 102 fails to consume items that promotes the growth of the specific microbe, the associated microbiome metric may decline (such as to represent an expected decrease in a quantity of the microbe).

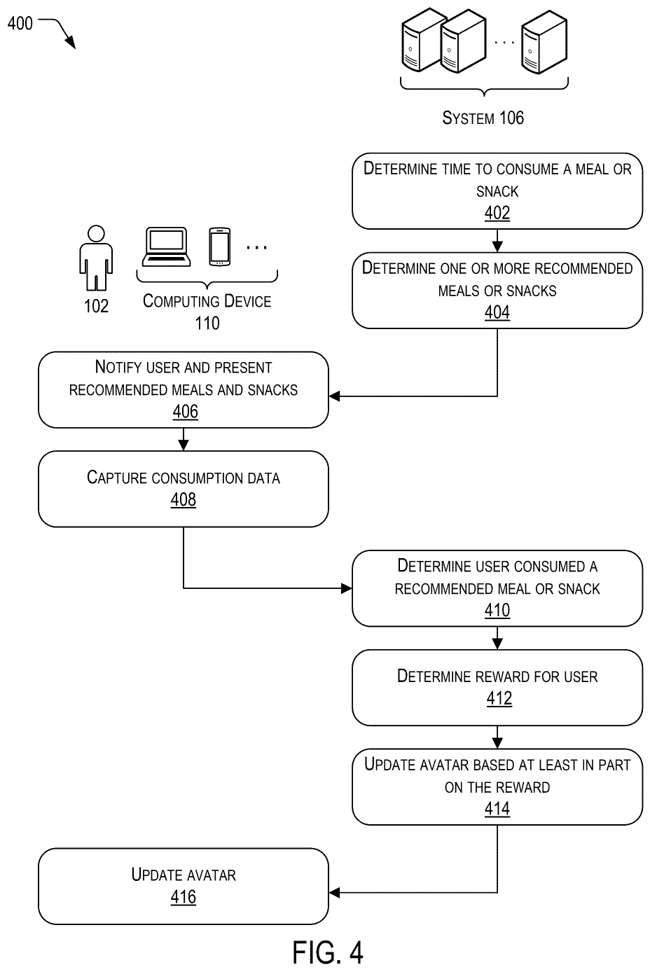

FIG. 4 is a flow diagram illustrating an example process 400 associated with the system 102 for recommending meals or snacks to assist in the improvement of the avatar, according to some implementations. As discussed herein, a user 102 may utilize an application hosted on a computing device 110 operating in conjunction with a cloud-based system 106 to maintain an avatar of visual representation of the microbiome of the user 102 over time that the user 102 may utilize to infer an overall status or health of their microbiome.

At 402, the system may determine it is time to consume a meal or snack. For instance, the system 106 may determine it is time to consume a meal or snack based on a period of time since their last meal or snack or based on the current time of day. In some cases, the user 102 may select or input particular times of day at which the user 102 desires to consume a meal or snack.

At 404, the system 106 may generate, such as based at least in part on the one or more food items, a nutritional value of each food item, diet indicated by the user 102, and the like for the user 102 at the determined meal or snack time. For example, the system 106 may generate two recipes or options for each meal that the user 102 normally consumes at a specified time. In some cases, the user 102 may select which meals on which weekdays the participant typically consumes. As an illustrative example, the user 102 may indicate they consume breakfast and dinner each day of the week but lunch only on Saturday. Similarly, the user 102 may indicate that on Wednesdays, user 102 works late and consumes a third meal after dinner. The system may then generate one or more meal plan options for the user 102 for each meal indicated during the given period of time.

At 406, the system 106 may send the one or more recommendations or meal plan options to a user device 110 associated with the user 102. In some cases, the system 106 may provide a health food score or microbiome score for each meal option presented.

At 408, the user 102 may, via an application hosted on the computing device 110, capture consumption data associated with a meal or snack (e.g., solid or liquid) the user 102 is preparing to consume. As discussed above, the user 102 may capture image data of the meal or snack via the computing device 110. As another example, the user may select the ingredients and/or quantity via a drop-down list provided by the application hosted on the computing device 110 or the user 102 may enter the ingredients and/or quantity a user input interface.

At 410, the system 106 may receive the consumption data (e.g., the image data representing the meal or snack) from the user 102 and determine nutritional data based at least in part on the consumption data. In some cases, the system 106 may be configured to process the consumption data received from the user 102 to determine ingredients, nutritional data associated with the ingredients, and/or other attributes or characteristics of the meal or snack being consumed by the user 102.

In some cases, the system 106 may utilize the one or more machine learning models to determine ingredients, nutritional data associated with the ingredients, and/or other attributes or characteristics of the meal or snack being consumed by the user 102. For instance, the one or more machine learning models may be configured to segment image data associated with a meal, such as a photograph captured by the user 102 using the computing device 110. The one or more machine learning models may then classify each item or object within the segmented image data to determine the type or identity of the ingredients, quantity or volume of each ingredient, and/or the like. The one or more machine learning models may utilize the classified ingredients as well as the quantity or volume to determine nutritional data associated with the meal represented in the image data provided by the user 102.

At 412, the system 106 may determine one or more microbiome metrics based at least in part on the nutritional data. For example, the system 106 may determine an effect of the nutritional data to one or more microbes either known to be present in a microbiome of the user 102 or commonly present in the microbiomes of individuals. For example, the system 106 may apply a heuristic, algorithmic, and/or other approach to determining the effect of the nutritional data on each microbe and/or the microbiome of the user 102 and/or the microbiome of a typical individual. In some specific examples, the system 106 may utilize one or more machine learned models trained to receive nutritional data as input and to output the microbiome metrics.

At 414, the system may update the status/appearance of the avatar or visual representations of the microbiome of the user 102 based at least in part on the microbiome metrics and, at 416, the computing device 110 may present the updated avatar on the display. For instance, as discussed above, the ingredients, quantities or amounts of each ingredient, together with a known effect of each ingredient on an individual's microbiome may be utilized to determine an expected positive or negative transformation of the microbes in the microbiome of the user 102 as represented by the microbiome metrics. The positive or negative transformation may be translated by the system 106 into a visible change to the status of the avatar that may be viewed by the user 102 on a display of the computing device 110.

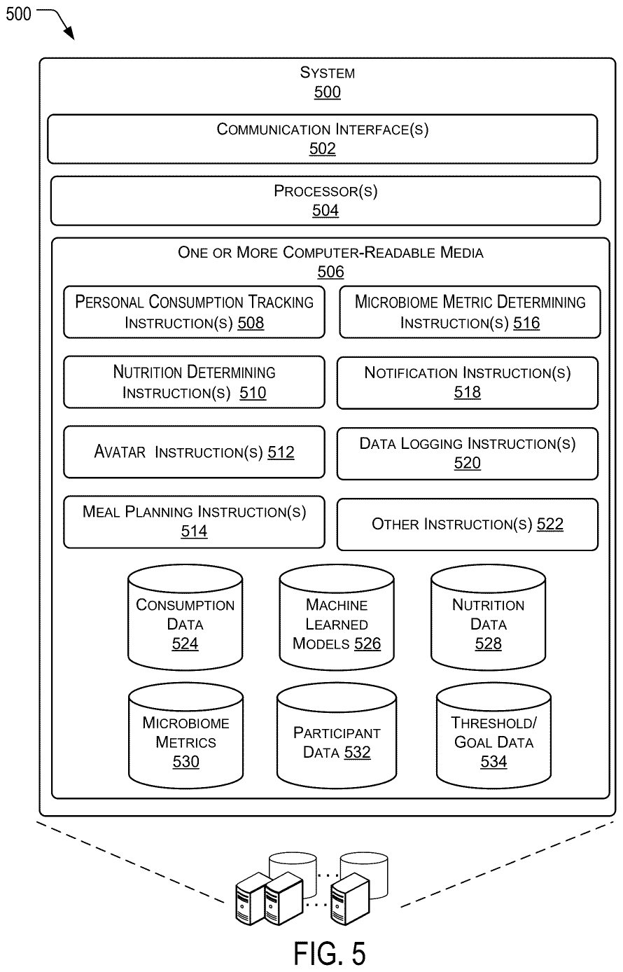

FIG. 5 is an example system 500 for providing a microbiome avatar or visual representation according to some implementations. The system 500 can include one or more communication interface(s) 502 that enable communication between the system 500 and one or more user devices associated with one or more participants. For instance, the communication interface(s) 502 can facilitate communication with other proximate sensor systems and/or other facility systems. The communications interfaces(s) 502 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

The system 500 may include one or more processors 504 and one or more computer-readable media 506. Each of the processors 504 may itself comprise one or more processors or processing cores. The computer-readable media 506 is illustrated as including memory/storage. The computer-readable media 506 may include volatile media (such as random access memory (RAM)) and/or nonvolatile media (such as read only memory (ROM), Flash memory, optical disks, magnetic disks, and so forth). The computer-readable media 506 may include fixed media (e.g., RAM, ROM, a fixed hard drive, and so on) as well as removable media (e.g., Flash memory, a removable hard drive, an optical disc, and so forth). The computer-readable media 506 may be configured in a variety of other ways as further described below.

Several modules such as instructions, data stores, and so forth may be stored within the computer-readable media 506 and configured to execute on the processors 504. For example, as illustrated, the computer-readable media 506 stores personal consumption tracking instructions 508, nutrition determining instructions 510, avatar instructions 512, meal planning instructions 514, microbiome metric determining instructions 516, notification instructions 518, data logging instruction 520 as well as other instructions 522, such as an operating system. The computer-readable media 606 may also be configured to store data, such as consumption data 524, machine learned models 526, nutrition data 528, microbiome metrics 530, participant data 532, and threshold/goal/target data 534 as well as other data.

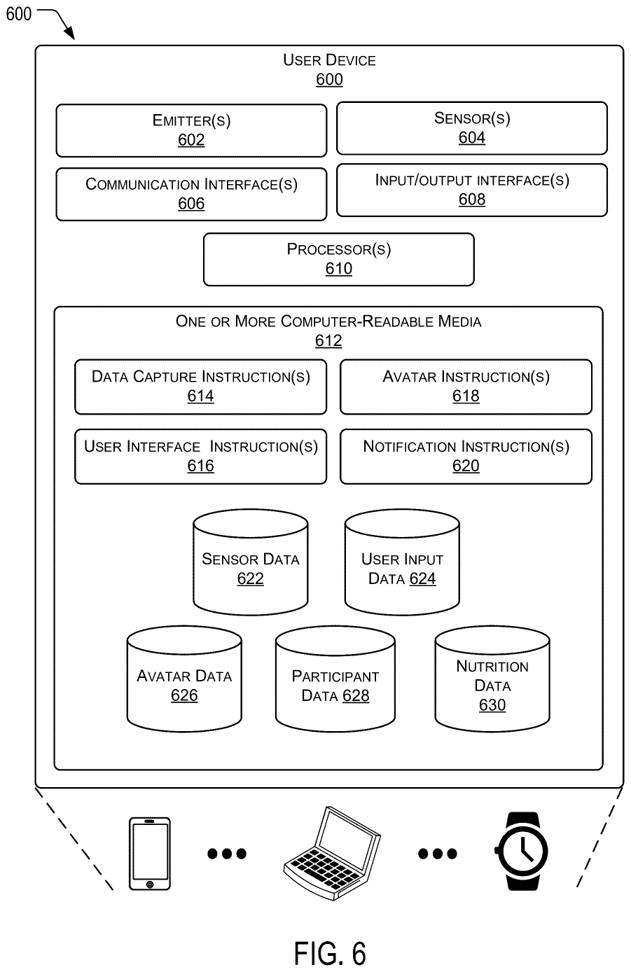

FIG. 6 is an example user device 600 that may implement the techniques described herein according to some implementations. In some cases, the user device 600 may be a hand-held electronic device equipped with sensors, a user interface, and one or more hosted applications. In some examples, the user device 600 may be implemented as a hand-held device in communication with a cloud-based system for providing and displaying one or more microbiome avatars, as discussed herein.

In some examples, the user device 600 may include one or more emitters 602. The emitters 602 may be mounted on an exterior surface of the user device 600 in order to output illumination or light into a physical environment, body feature (e.g., a wrist in the case of a blood pressure monitor or the like), or the like. The emitters 602 may include, but are not limited to, visible lights emitters, infrared emitters, ultraviolet light emitters, LIDAR systems, and the like. In some cases, the emitters 602 may output light in predetermined patterns, varying wavelengths, or at various time intervals (e.g., such as pulsed light).

The user device 600 may also include one or more sensors 604. The sensor 604 may include image sensors, depth sensors, motion sensors, position sensors, health sensors, wearable sensors, body sensors, and the like. For example, the sensors 604 may include image devices, spectral sensors, IMUs, accelerometers, gyroscopes, depth sensors, infrared sensors, GPS systems, blood sugar sensor, and/or the like. In various examples, the sensors 604 may be utilized to capture image data of meals or snacks the user is preparing to consume.

The user device 600 may also include one or more communication interfaces 606 configured to facilitate communication between one or more networks, one or more cloud-based system(s), and/or one or more mobile or user devices. In some cases, the communication interfaces 606 may be configured to send and receive data with, for instance, the cloud-based system as discussed above. The communications interfaces(s) 606 may enable Wi-Fi-based communication such as via frequencies defined by the IEEE 802.11 standards, short range wireless frequencies such as Bluetooth, cellular communication (e.g., 2G, 3G, 4G, 4G LTE, 5G, etc.), satellite communication, dedicated short-range communications (DSRC), or any suitable wired or wireless communications protocol that enables the respective computing device to interface with the other computing device(s).

In the illustrated example, the user device 600 also includes an input and/or output interface 608, such as a projector, a virtual environment display, a traditional 2D display, buttons, knobs, and/or other input/output interfaces. For instance, in one example, the interfaces 608 may include a flat display surface, such as a touch screen configured to allow a user of the device 600 to consume content (such as the microbiome avatar) and to provide feedback in the form of touch inputs. In one example, the avatars may be touch sensitive or react to a touch input on the touch enabled display to appear responsive to the user interactions.

The user device 600 may also include one or more processors 610, such as at least one or more access components, control logic circuits, central processing units, or processors, as well as one or more computer-readable media 612 to perform the function associated with the virtual environment. Additionally, each of the processors 610 may itself comprise one or more processors or processing cores.

Depending on the configuration, the computer-readable media 612 may be an example of tangible non-transitory computer storage media and may include volatile and nonvolatile memory and/or removable and non-removable media implemented in any type of technology for storage of information such as computer-readable instructions or modules, data structures, program modules or other data. Such computer-readable media may include, but is not limited to, RAM, ROM, EEPROM, flash memory or other computer-readable media technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, or any other medium that can be used to store information and which can be accessed by the processors 610.

Several modules such as instructions, data stores, and so forth may be stored within the computer-readable media 612 and configured to execute on the processors 610. For example, as illustrated, the computer-readable media 612 may store data capture and/or scanning instructions 614, user interface instructions 616, avatar instructions 618, notification instructions 620, as well as other instructions. The computer-readable media 612 may also store data such as sensor data 622, user input data 624, avatar data 626, participant data 628, nutrition data 630, and the like.

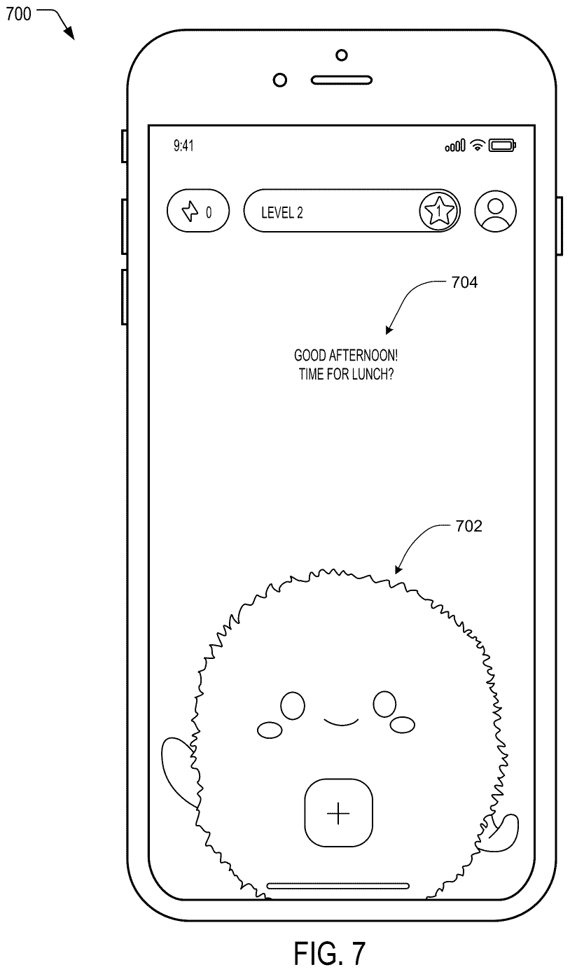

FIG. 7 is an example pictorial diagram illustrating an example user interface 700 associated with a microbiome avatar 702 according to some implementations. For instance, in the current example, a user device, such as a smartphone, tablet, or other electronic device having a display, may present the user interface 700 including the avatar 702. In the current example, the avatar 702 appears happy and energic. This may be a result of healthy eating associated with one or more prior meals, such as last night's dinner. In this example, the system may provide a notification 704 to the user that it is time for lunch and the presence of the avatar 702 may help to encourage the user to continue to regularly consume a healthy diet.

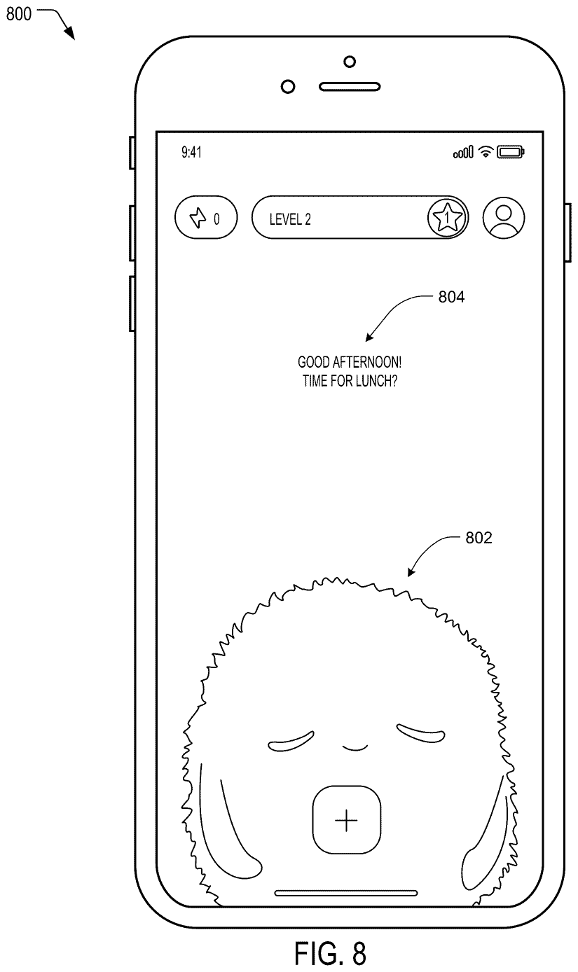

FIG. 8 is an example pictorial diagram illustrating an example user interface 800 associated with a microbiome avatar 802 according to some implementations. For instance, in the current example, a user device, such as a smartphone, tablet, or other electronic device having a display, may present the user interface 800 including the avatar 802. In the current example, the avatar 802 appears sleepy or tired. This may be a result of poor eating at prior meals, such as last nights'dinner. However, in this example, the avatar 802 may appear tired as it is time for a meal, such as lunch. In this manner, the system may provide a notification 804 to the user that it is time for lunch and the user may infer the need to consume healthy food or drinks quickly as the avatar 802 is sleepy.

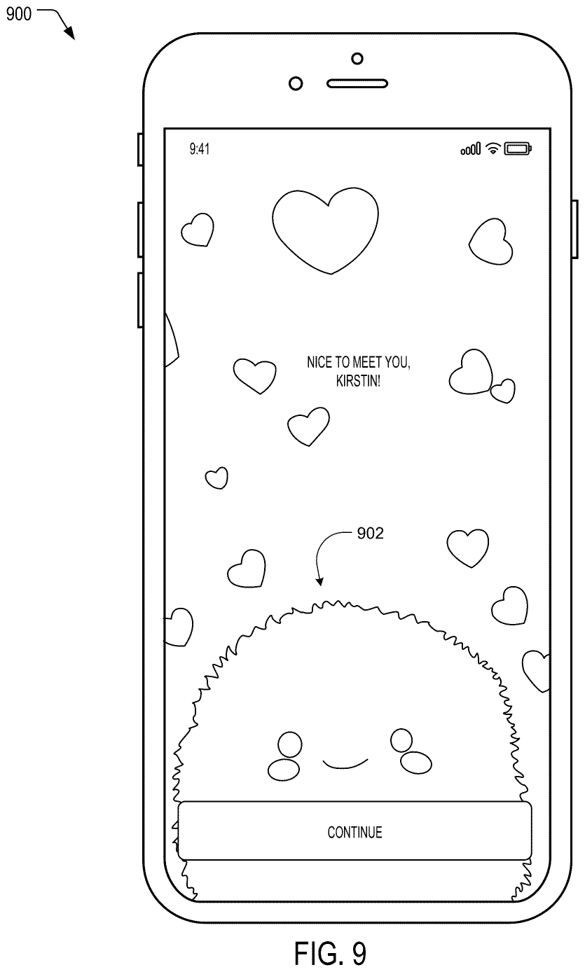

FIG. 9 is an example pictorial diagram illustrating an example user interface 900 associated with a microbiome avatar 902 according to some implementations. For instance, in the current example, a user device, such as a smartphone, tablet, or other electronic device having a display, may present the user interface 900 including the avatar 902. In the current example, the user may have recently created an account with the system and the user may be introduced or just have finished generating or creating their avatar 902. For instance, in this example, the avatar 902 may be at an initial stage, lifecycle or level (e.g., at level zero). Accordingly, the avatar 902 may be in a basic form, for instance, the avatar 902 lacks arms in this example, unlike the avatar 702 and 802 of FIGS. 7 and 8 in which the avatars are level 2 or have advanced in response to healthy eating by the user. Accordingly, the avatars 702 and 802 have arms.

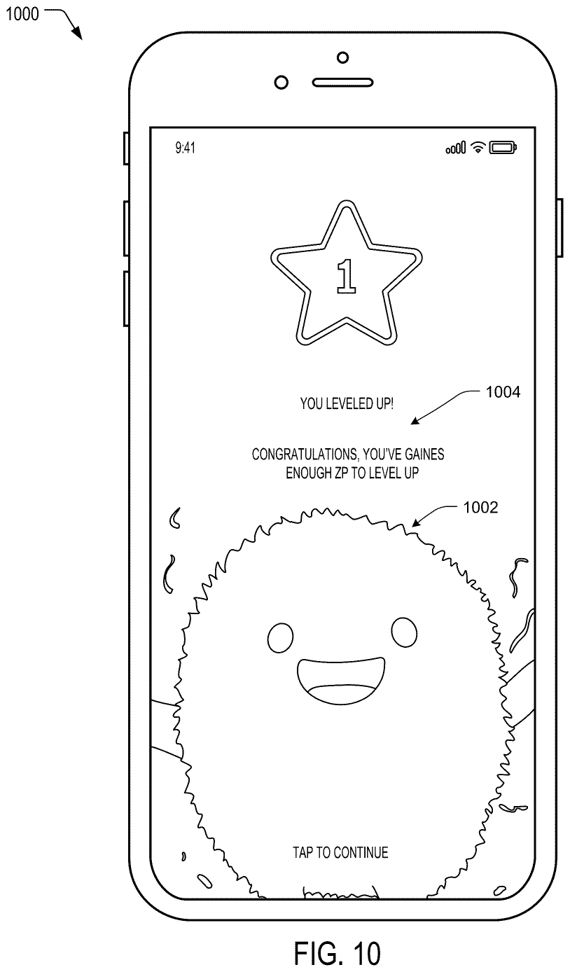

FIG. 10 is an example pictorial diagram illustrating an example user interface 1000 associated with a microbiome avatar 1002 according to some implementations. For instance, in the current example, a user device, such as a smartphone, tablet, or other electronic device having a display, may present the user interface 1000 including the avatar 1002. In the current example, the user may have recently consumed a healthy meal that had a positive effect on the user's microbiome and resulted in, for instance, a cumulation of positive change in their microbiome to cause the avatar 1002 representing the user's microbiome to level up or advance. Accordingly, the user interface 1000 is providing a notification 1004 that the avatar 1002 has levelled up or otherwise advanced, the avatar 1002 is happy and energic, and the appearance of the avatar 1002 may have changed. For instance, in the current example, the avatar 1002 includes arms and its fur appears longer than the avatar 902 of FIG. 9, which illustrated the avatar at the time of creation. In this manner, the user may infer how their healthy eating has improved their overall gut health and microbiome in an easily understandable way.

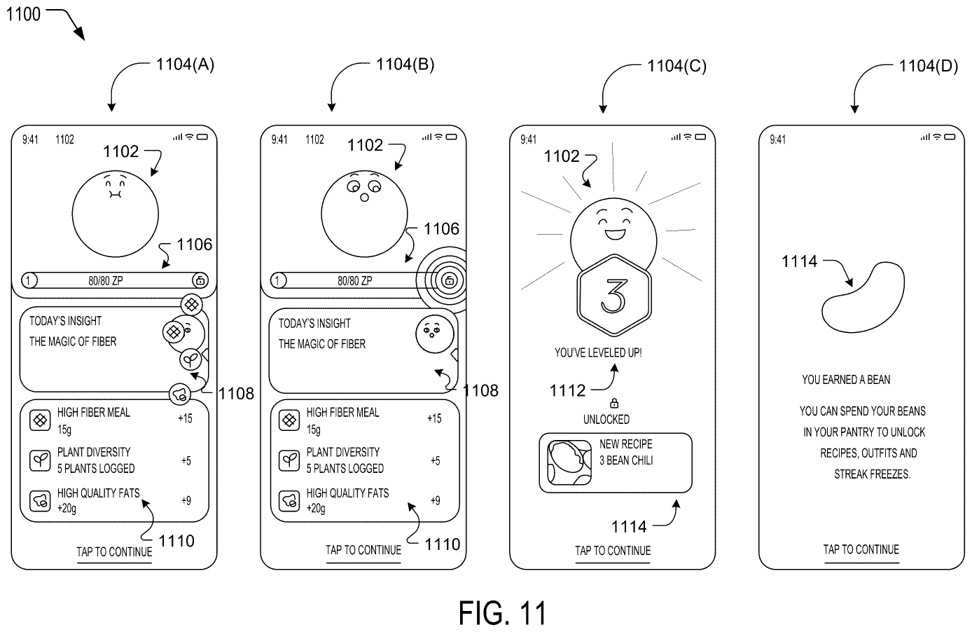

FIG. 11 is a series of example pictorial diagrams 1100 illustrating example user interfaces 1104(A)-(D) associated with a microbiome avatar 1102 according to some implementations. In the current example, the user interfaces 1004 illustrate a progress bar 1106 associated with the user's healthy eating and the status of the avatar 1102. The user interfaces 1104(A) and 1104(B) also includes an insight or notification 1108 to provide the user with recommendations, tips, and the like to improve the eating habits of the user, such as the benefits of consuming fiber in this example.

In the current example, the user has also just logged or input a meal (such as by uploading or capturing an image of the meal, as discussed herein). Accordingly, the user interface 1104(A) shows that the meal was high in fiber, had plant diversity, and included high quality fats via the notification, generally indicated by 1110. In this example, the avatar 1102 earned progress for the user consuming the meal including the fiber, plant diversity, and high quality fats. For instance, the avatar 1102 earned 15 points (or microbiome metrics) for the fiber, 5 points (or microbiome metrics) for the plant diversity, and 9 points (or microbiome metrics) for the fats. The additional points (or microbiome metrics) have caused the progress bar 1106 to complete, as illustrated in user interface 1104(B). Consequently, the user interface 1104(C) provides a notification 1112 that the avatar 1102 has leveled up or otherwise progressed. Again, the avatar 1102 is happy and energetic illustrating the effects of positive eating on the user's microbiome. In some cases, to encourage the user to eat healthy, as the avatar 1102 progresses, the system may provide rewards, generally indicated by 1114, such as new recipes and the like. In some cases, such as the user interface 1104(D), the rewards 1114 may be in the form a bean or other digital currency that the user may spend via the system.

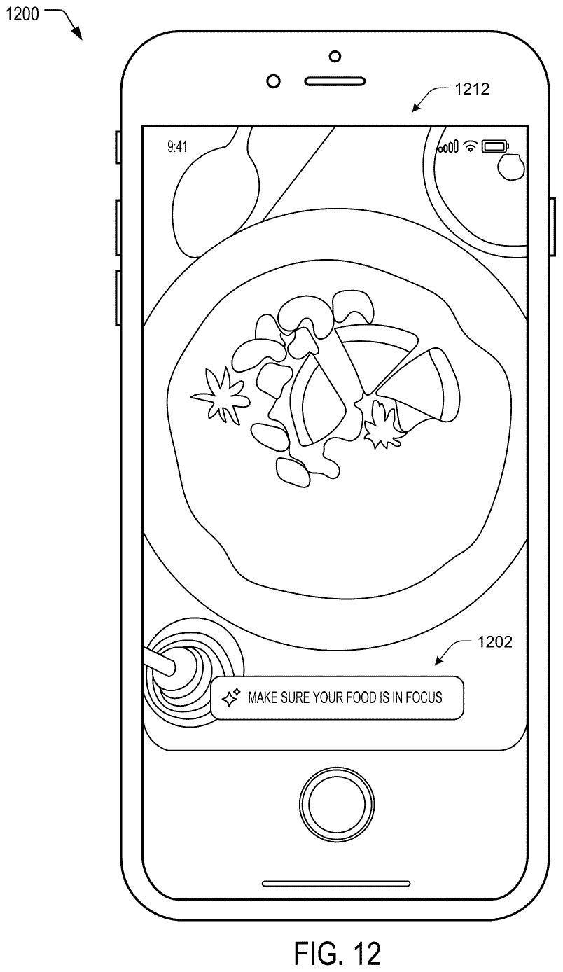

FIG. 12 is an example pictorial diagram illustrating an example user interface 1200 associated with capturing consumption data for use in maintaining a microbiome avatar according to some implementations. For instance, in the current example, a user device, such as a smartphone, tablet, or other electronic device having a display, may present the user interface 1200 usable to capture, for instance, image data of a meal that the user is preparing to consume. In some cases, the user interface 1200 may assist the user in making sure a quality capture event occurs, such as by providing tips and suggestions 1202 to improve the capture event. In some cases, such as when the meal is on multiple settings, the system may allow for a video type capture and provide scanning instructions via the suggestion window 1202.

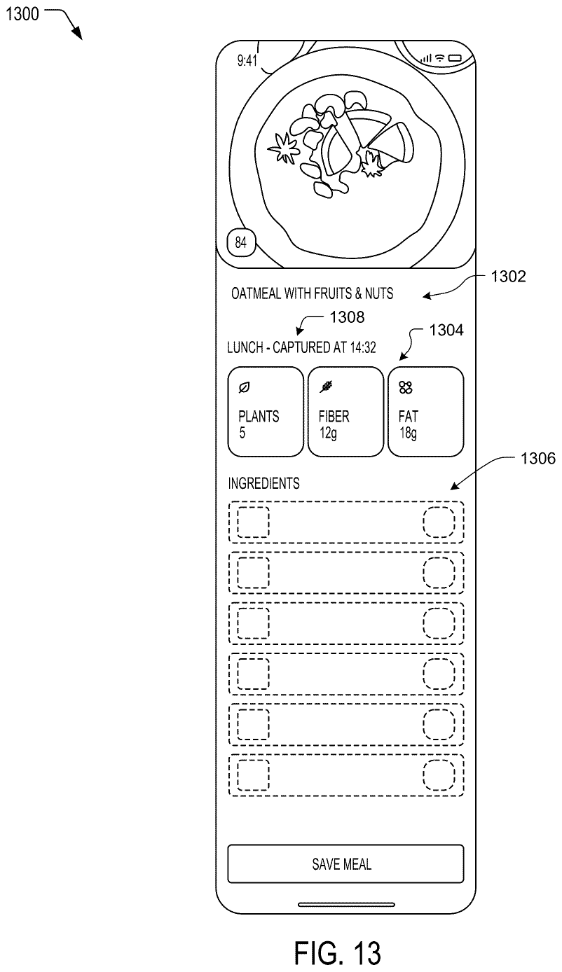

FIG. 13 is an example pictorial diagram illustrating an example user interface 1300 associated with an output or response to capturing consumption data for use in maintain a microbiome avatar according to some implementations. For instance, in the current example, a user device, such as a smartphone, tablet, or other electronic device having a display, may present the user interface 1300 usable to capture, for instance, image data of a meal that the user is preparing to consume. In some cases, a system, such as a cloud-based system, may process the image data captured with respect to FIG. 12 above. Once the nutritional data is determined, the user interface 1300 may be utilized to present or display the nutritional data to the user. For instance, the user interface 1300 may present a label 1302 for the meal together with a list of highlighted nutritional facts or data 1304. For example, in the current illustration, the user interface 1300 may highlight that the meal includes 5 types of plants, 12 grams of fiber, and 18 grams of healthy fats. The system may also provide a list of other detected ingredients or nutritional data as a more detailed list 1306. The system has also categorized and time stamped the meal, generally indicated by 1308.

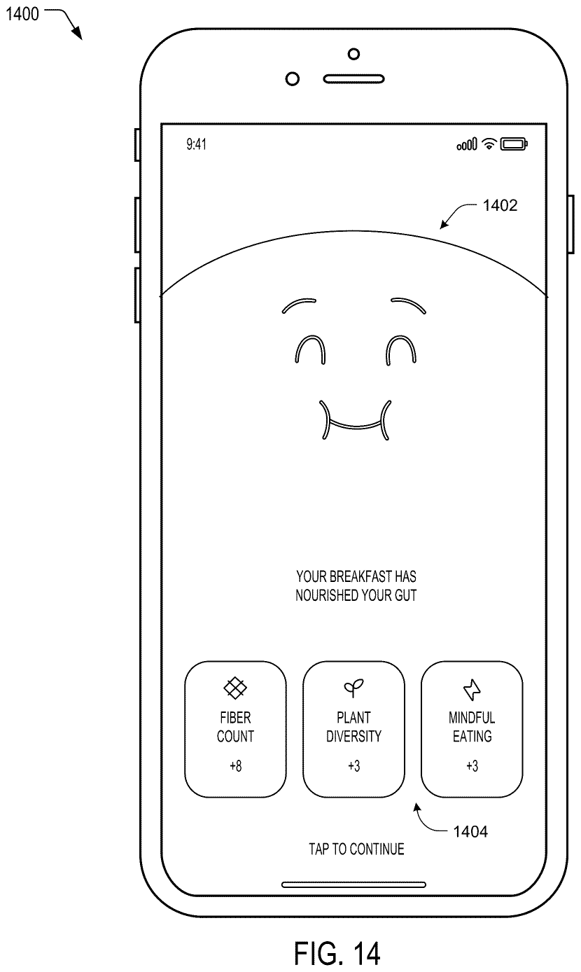

FIG. 14 is an example pictorial diagram illustrating an example user interface 1400 associated with a microbiome avatar 1402 after a meal is logged according to some implementations. For instance, in the current example, a user device, such as a smartphone, tablet, or other electronic device having a display, may present the user interface 1400 including the avatar 1402. In the current example, the user may have recently consumed a healthy meal, such as the breakfast of FIGS. 12 and 13, that had a positive effect on the user's microbiome. Accordingly, the avatar 1402 is happy and the system may provide a notification 1404 related to particular microbiome metrics that the meal provided. For example, the breakfast included high fiber, positive plant diversity, and the user's act of carefully recording this healthy breakfast demonstrates thoughtfulness about their dietary intake and so could be considered mindful eating.

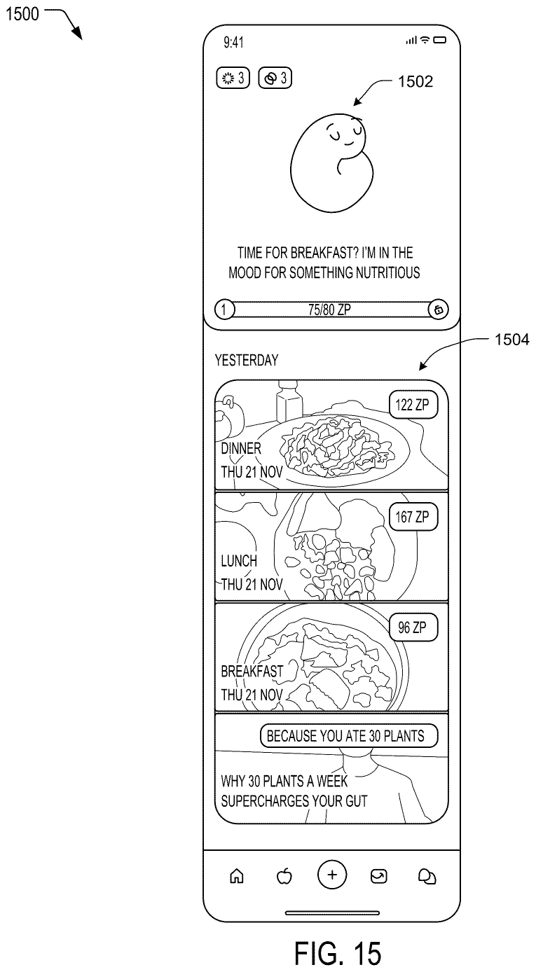

FIG. 15 is an example pictorial diagram illustrating an example user interface 1500 associated with a microbiome avatar 1502 according to some implementations. For instance, in the current example, a user device, such as a smartphone, tablet, or other electronic device having a display, may present the user interface 1500 including the avatar 1502. In the current example, the avatar 1502 appears happy and energic. This may be a result of healthy eating associated with one or more prior meals, such as yesterday's meals which may be presented as a log 1504. In the current example, the log 1504 may be partitioned by meals (and/or snacks) such as based on time stamps on when the user captured the consumption data associated with each meal. In some cases, each meal may be selectable to provide nutritional data associated with that meal.

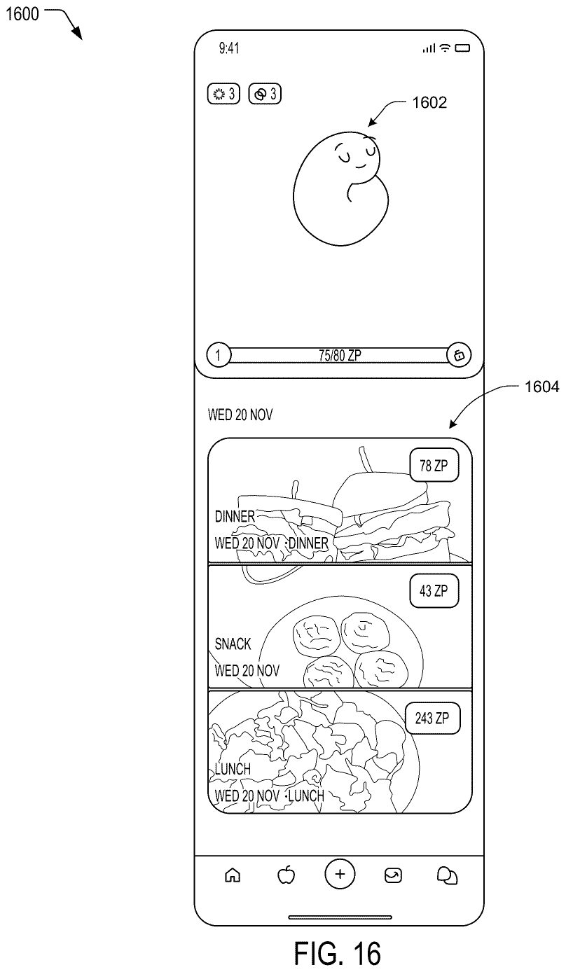

FIG. 16 is an example pictorial diagram illustrating an example user interface 1600 associated with a microbiome avatar 1602 according to some implementations. For instance, in the current example, a user device, such as a smartphone, tablet, or other electronic device having a display, may present the user interface 1600 including the avatar 1602. In the current example, the avatar 1602 appears happy and energic. This may be a result of healthy eating associated with one or more prior meals, such as yesterday's meals which may be presented as a log 1604. In the current example, the log 1604 may be partitioned by meals (and/or snacks) such as based on time stamps on when the user captured the consumption data associated with each meal. In some cases, each meal may be selectable to provide nutritional data associated with that meal.

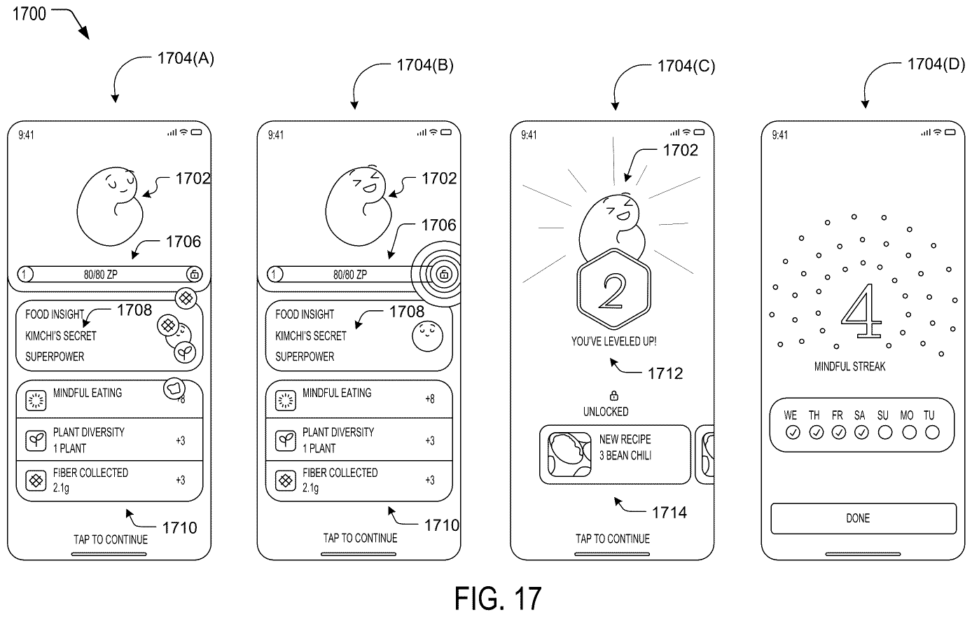

FIG. 17 is a series of example pictorial diagrams 1700 illustrating example user interfaces 1704(A)-(D) associated with a microbiome avatar 1702 according to some implementations. In the current example, the user interfaces 1704 illustrate a progress bar 1706 associated with the user's healthy eating and the status of the avatar 1702. The user interfaces 1704(A) and 1704(B) also includes an insight or notification 1708 to provide the user with recommendations, tips, and the like to improve the eating habits of the user, such as the benefits of consuming kimchi or other fermented products in this example.

In the current example, the user has also just logged or input a meal (such as by uploading or capturing an image of the meal, as discussed herein). Accordingly, the user interface 1704(A) shows that the meal was considered mindful eating, had plant diversity, and was high in fiber, via the notification, generally indicated by 1710. In this example, the avatar 1702 earned progress for the user consuming the meal including the fiber, plant diversity, and for their mindful eating, as discussed above. The additional points (or microbiome metrics) have caused the progress bar 1706 to complete, as illustrated in user interface 1704(B). Consequently, the user interface 1704(C) provides a notification 1712 that the avatar 1702 has leveled up or otherwise progressed. Again, the avatar 1702 is happy and energetic illustrating the effects of positive eating on the user's microbiome. In some cases, to encourage the user to eat healthy, as the avatar 1702 progresses, the system may provide rewards, generally indicated by 1714, such as new recipes and the like. In some cases, such as the user interface 1704(D), the system may provide the user with additional notices, such as to re-enforce consistency in healthy eating habits (e.g., in this example, mindful eating).

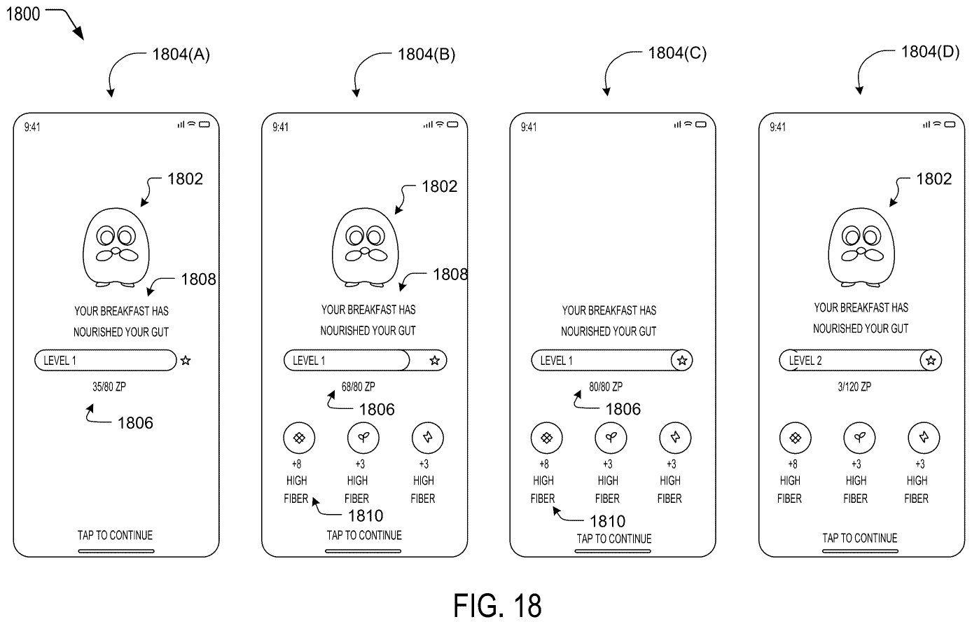

FIG. 18 is a series of example pictorial diagrams 1800 illustrating example user interfaces 1804(A)-(D) associated with a microbiome avatar 1802 according to some implementations. In the current example, the user interfaces 1804 illustrate a progress indicator 1806 associated with the user's healthy eating and the status of the avatar 1802. In the current example, the user has logged or input a meal (such as by uploading or capturing an image of the meal, as discussed herein). Accordingly, the user interface 1804(B) shows that the meal was high in three types of fiber, via the notification, generally indicated by 1810. In this example, the avatar 1802 earned progress for the user consuming the meal including the three types of fiber, as discussed above. The additional points (or microbiome metrics) have caused the progress indicator 1806 to complete in user interface 1804(C). Consequently, the user interface 1704(D) illustrates a change in the avatar 1802 (e.g., the avatar 1802 is full and happy).

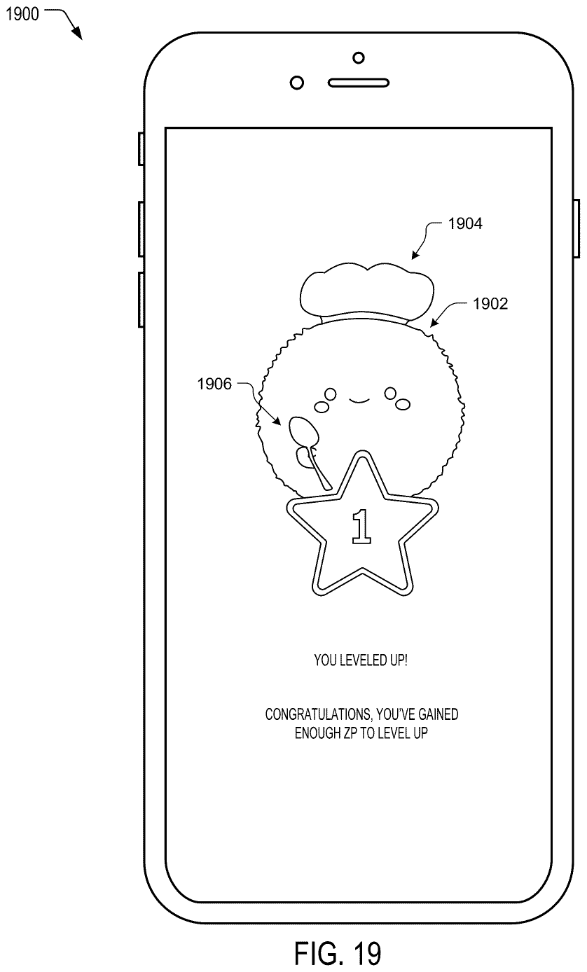

FIG. 19 is an example pictorial diagram illustrating an example user interface 1900 associated with a microbiome avatar 1902 according to some implementations. For instance, in the current example, a user device, such as a smartphone, tablet, or other electronic device having a display, may present the user interface 1900 including the avatar 1902. In the current example, the avatar 1902 has changed their outfit to accommodate the situational based experiences of the user. For example, after the user consumes a healthy meal the avatar may be updated to include an outfit or other accessory that shows that the avatar 1902 has experienced a positive outcome (e.g., the individual's microbiome is benefiting form the meal). As an example, the avatar may include sporting accessories (e.g., a jersey, a racket, vizor, ball, cleats, dumbbells, and/or the like) to illustrate to the user that the avatar is energized, healthy, and ready to go.

In the current example, the avatar 1902 is illustrated wearing a chef hat 1904 and holding a spoon 1906. For instance, in the current example, the user may be requesting a recipe that will nourish their microbiome and the avatar 1902 has changed into a chef's outfit to assist the user with preparing the meal as the system provides recommended recipes, meals, ingredients, and/or the like.

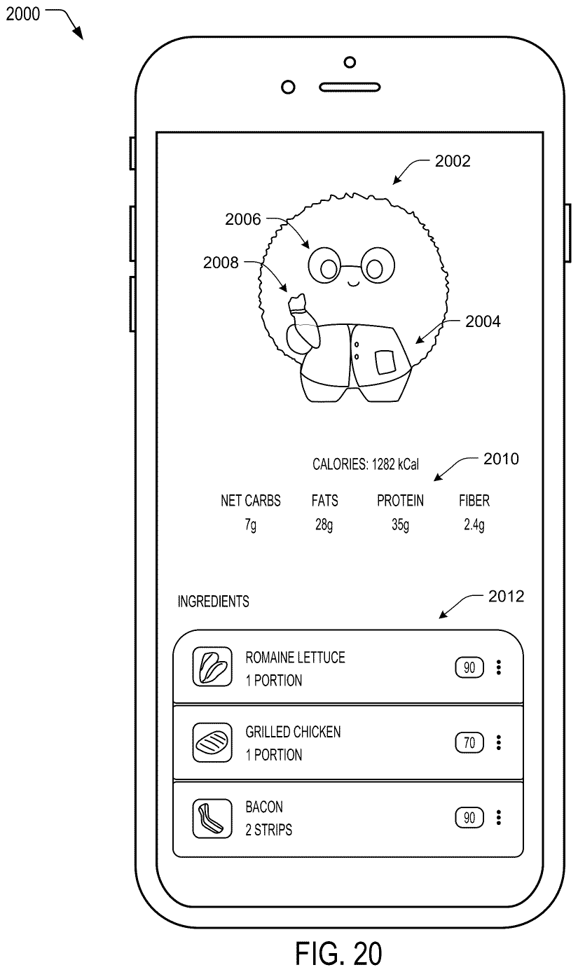

FIG. 20 is an example pictorial diagram illustrating an example user interface 2000 associated with a microbiome avatar 2002 according to some implementations. For instance, in the current example, a user device, such as a smartphone, tablet, or other electronic device having a display, may present the user interface 2000 including the avatar 2002. In the current example, the avatar 2002 has changed their outfit to accommodate the situational based experiences of the user. For example, after the user consumes a meal the avatar 2002 may be used to explain the nutritional or microbiome nourishing components of the meal. In these situations, the avatar 2002 may be updated to include an outfit or other accessory associated with educational professions or occupations to cause the user to infer that the user is about learn. For instance, in the current example, the avatar 2002 has dressed in a lab coat 2004, glasses 2006, and is holding a test tube 2008. At the same time, the user interface 2000 has presented the user with a list of microbiome metrics 2010 and ingredients 2012 associated with a meal that the user has reported to the system.

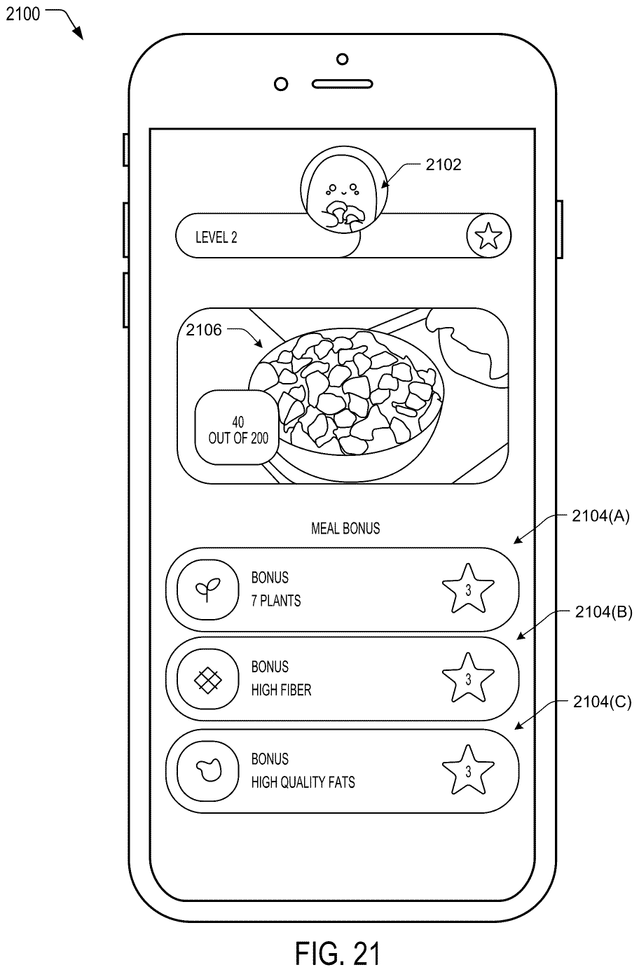

FIG. 21 is an example pictorial diagram illustrating an example user interface 2100 associated with a microbiome avatar 2102 according to some implementations. For instance, in the current example, a user device, such as a smartphone, tablet, or other electronic device having a display, may present the user interface 2100 including the avatar 2102. In the current example, the avatar 2102 is holding broccoli, such as responsive to the nutritional data or ingredients of a meal including broccoli. In the current example, the user interface 2100 is also illustrating various microbiome metrics 2104(A)-(C) that are associated with a meal, generally indicated by 2106. For example, the microbiome metric 2104(A) may show a positive 3 value to consuming seven plants, a positive 10 value for consuming high fiber, and a positive 3 value for consuming high quality fats. In this manner, the microbiome metrics may represent an expected change in the health of the microbiome of the user in response to consuming the meal 2106. As some illustrative examples, the microbiome metrics may include, but are not limited to, an expected microbiome improvement due to high fiber, an expected microbiome improvement due to high plant diversity, an expected microbiome improvement due to high quality fats; an expected microbiome deterioration due to low quality fats, an expected microbiome improvement due to high polyphenols, expected microbiome deterioration due to additives or preservatives, and/or an expected microbiome deterioration due to high level of processing. In some cases, the microbiome metrics may be associated with a meal, item, ingredient of a meal, or other nutritional aspect of an item consumed by a user.

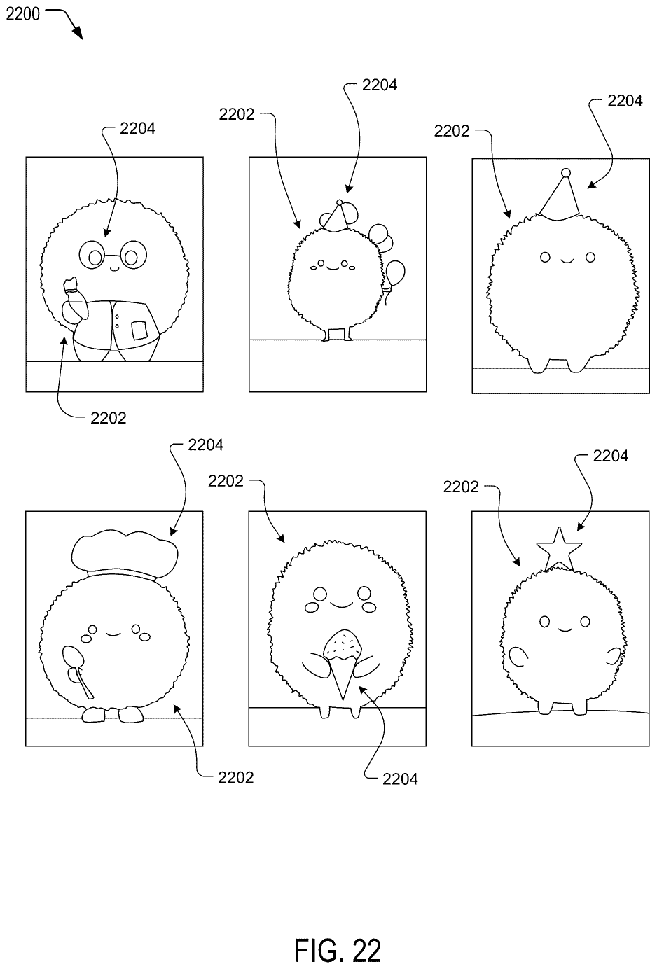

FIG. 22 is a series of example pictorial diagrams 2200 illustrating an example a microbiome avatar 2202 with various accessories, generally indicated by 2204, according to some implementations. For instance, in the various illustrated diagrams 2200, the avatar 2202 may have glasses, a lab coat, a test tube, a birthday hat, balloons, a chefs hat, a spoon or utensil, an ice cream cone, and a star. In other examples, the avatar 2202 may have other accessories than those illustrated in FIG. 22. For example, the avatar may have gym or workout equipment, sporting accessories, travel accessories, shopping accessories, lifestyle accessories, occupational related accessories, and/or the like. In some specific examples, the accessories may be based at least in part on a physical location of the user device. For instance, if the user device is present on a beach, the avatar 2102 may wear a bathing suit and hold a surfboard and appear ready to swim when the microbiome is healthy but a sunhat and sunglasses and appear tired when the microbiome is unhealthy.

Although the discussion above sets forth example implementations of the described techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

### EXAMPLE CLAUSES

A. A method comprising: receiving consumption data associated with a meal from a user device associated with a user; determining, based at least in part on the consumption data, one or more microbiome metrics associated with the meal; determining, based at least in part on the one or more microbiome metrics associated with the meal, a status of an avatar representing a health of a microbiome of the user; determining, based at least in part on the status, a first visual characteristic of the avatar, an object associated with the avatar, or an environment associated with the avatar; and causing the first visual characteristic to appear with the avatar on a display of the user device.
B. The method of A, wherein the consumption data includes image data of the meal.
C. The method of A, further comprising: determining, based at least in part on the consumption data, nutritional data associated with the meal; and wherein determining the one or more microbiome metrics is based at least in part on the nutritional data.
D. The method of C, wherein determining the nutritional data associated with the meal further comprises inputting the consumption data into one or more machine learning models trained to segment, classify, and characterize the consumption data and to output the nutritional data.
E. The method of A, wherein determining the one or more microbiome metrics further comprises inputting the consumption data into one or more machine learning models trained to segment, classify, and characterize the consumption data and to output the one or more microbiome metrics.
F. The method of A, wherein determining the status of the avatar representing the health of the microbiome of the user is based at least in part on a sliding window of time.
G. The method of A, further comprising: receiving personalization data associated with the user; and generating, based at least in part on the personalization data, the avatar.
H. The method of A, further comprising providing at least one selectable option to allow the user to modify a second visual characteristic of the avatar, an object associated with the avatar, or an environment associated with the avatar.
I. The method of A, wherein the first visual characteristic is at least one of: a size of the avatar; a lifecycle or life stage of the avatar; an energy level of the avatar; a heath of the avatar; a mood of the avatar; an accessory associated with the avatar; an article of clothing worn by the avatar; a piece of equipment associated with the avatar; an activity performed by the avatar; or an environment associated with the avatar.
J. The method of A, wherein the one or more microbiome metrics associated with the meal comprise at least one of: expected microbiome improvement due to high fiber; expected microbiome improvement due to high plant diversity; expected microbiome improvement due to high quality fats; expected microbiome deterioration due to low quality fats; expected microbiome improvement due to high polyphenols; expected microbiome deterioration due to additives or preservatives; or expected microbiome deterioration due to high level of processing.
K. A system comprising: one or more processors; and one or more non-transitory computer readable media storing instructions executable by the one or more processors, wherein the instruction, when executed, cause the one or more processors to perform operations comprising: receiving consumption data associated with a meal from a user device associated with a user; determining, based at least in part on the consumption data, one or more microbiome metrics associated with the meal; determining, based at least in part on the one or more microbiome metrics associated with the meal, a visual characteristic of an avatar, an object associated with the avatar, or an environment associated with the avatar, representing a health of a microbiome of the user; and causing the visual characteristic and the avatar to appear on a display of the user device.
L. The system of K, wherein determining the status of the avatar is based at least in part on one or more cumulative values.
M. The system of K, wherein the operations further comprise determining, based at least in part on the consumption data, nutritional data associated with the meal; and wherein determining the one or more microbiome metrics is based at least in part on the nutritional data.
N. The system of M, further comprising sending a notification to the user device, the notification indicating: at least a portion of the nutritional data; or at least one of the one or more microbiome metrics.
O. One or more non-transitory computer readable media storing instructions executable by one or more processors, wherein the instruction, when executed, cause the one or more processors to perform operations comprising: receiving, from a user device, image data of a meal associated with a user of the user device; determining, based at least in part on the image data, one or more microbiome metrics associated with the meal; determining, based at least in part on the one or more microbiome metrics associated with the meal, a visual characteristic associated with presenting an avatar on a display, the avatar representing a health of a microbiome of the user; and causing the visual characteristic and the avatar to appear on the display.
P. The one or more non-transitory computer readable media of O, wherein the operations further comprise: determining, based at least in part on the consumption data, nutritional data associated with the meal; and wherein determining the one or more microbiome metrics is based at least in part on the nutritional data.
Q. The one or more non-transitory computer readable media of P, wherein the operations further comprise: logging the nutritional data and the meal; and providing the user access to the logged nutritional data.
R. The one or more non-transitory computer readable media of claim of O, wherein the operations further comprise: receiving personalization data associated with the user; and generating, based at least in part on the personalization data, the avatar.
Q. The one or more non-transitory computer readable media of R, wherein the personalization data includes a lab result of a test of the microbiome of the user.
T. The one or more non-transitory computer readable media O, wherein the operations further comprise: determining, based at least in part on the one or more microbiome metrics, a recommended recipe for the user; and causing the recommended recipe to be presented on a display of the user device.

While the example clauses described above are described with respect to one particular implementation, it should be understood that, in the context of this document, the content of the example clauses can also be implemented via a method, device, system, a computer-readable medium, and/or another implementation. Additionally, any of examples A-T may be implemented alone or in combination with any other one or more of the examples A-T.

### CONCLUSION

While one or more examples of the techniques described herein have been described, various alterations, additions, permutations and equivalents thereof are included within the scope of the techniques described herein. As can be understood, the components discussed herein are described as divided for illustrative purposes. However, the operations performed by the various components can be combined or performed in any other component. It should also be understood that components or steps discussed with respect to one example or implementation may be used in conjunction with components or steps of other examples.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

What is claimed is:

1. A method comprising:
receiving consumption data associated with a meal from a user device associated with a user;
determining, based at least in part on the consumption data, one or more microbiome metrics associated with the meal;
determining, based at least in part on the one or more microbiome metrics associated with the meal, a status of an avatar representing a health of a microbiome of the user;
determining, based at least in part on the status, a first visual characteristic of the avatar, an object associated with the avatar, or an environment associated with the avatar; and
causing the first visual characteristic to appear with the avatar on a display of the user device.

2. The method of claim 1, wherein the consumption data includes image data of the meal.

3. The method of claim 1, further comprising:
determining, based at least in part on the consumption data, nutritional data associated with the meal; and
wherein determining the one or more microbiome metrics is based at least in part on the nutritional data.

4. The method of claim 3, wherein determining the nutritional data associated with the meal further comprises inputting the consumption data into one or more machine learning models trained to segment, classify, and characterize the consumption data and to output the nutritional data.

5. The method of claim 1, wherein determining the one or more microbiome metrics further comprises inputting the consumption data into one or more machine learning models trained to segment, classify, and characterize the consumption data and to output the one or more microbiome metrics.

6. The method of claim 1, wherein determining the status of the avatar representing the health of the microbiome of the user is based at least in part on a sliding window of time.

7. The method of claim 1, further comprising:
receiving personalization data associated with the user; and
generating, based at least in part on the personalization data, the avatar.

8. The method of claim 1, further comprising providing at least one selectable option to allow the user to modify a second visual characteristic of the avatar, an object associated with the avatar, or an environment associated with the avatar.

9. The method of claim 1, wherein the first visual characteristic is at least one of:
a size of the avatar;
a lifecycle or life stage of the avatar;
an energy level of the avatar;
a heath of the avatar;
a mood of the avatar;
an accessory associated with the avatar;
an article of clothing worn by the avatar;
a piece of equipment associated with the avatar;
an activity performed by the avatar; or
an environment associated with the avatar.

10. The method of claim 1, wherein the one or more microbiome metrics associated with the meal comprise at least one of:
expected microbiome improvement due to high fiber;
expected microbiome improvement due to high plant diversity;
expected microbiome improvement due to high quality fats;
expected microbiome deterioration due to low quality fats;
expected microbiome improvement due to high polyphenols;
expected microbiome deterioration due to additives or preservatives; or
expected microbiome deterioration due to high level of processing.

11. A system comprising:
one or more processors; and
one or more non-transitory computer readable media storing instructions executable by the one or more processors, wherein the instruction, when executed, cause the one or more processors to perform operations comprising:
receiving consumption data associated with a meal from a user device associated with a user;
determining, based at least in part on the consumption data, one or more microbiome metrics associated with the meal;
determining, based at least in part on the one or more microbiome metrics associated with the meal, a visual characteristic of an avatar, an object associated with the avatar, or an environment associated with the avatar, representing a health of a microbiome of the user; and
causing the visual characteristic and the avatar to appear on a display of the user device.

12. The system of claim 11, wherein determining the status of the avatar is based at least in part on one or more cumulative values.

13. The system of claim 11, wherein the operations further comprise:
determining, based at least in part on the consumption data, nutritional data associated with the meal; and
wherein determining the one or more microbiome metrics is based at least in part on the nutritional data.

14. The system of claim 13, further comprising sending a notification to the user device, the notification indicating:
at least a portion of the nutritional data; or
at least one of the one or more microbiome metrics.

15. One or more non-transitory computer readable media storing instructions executable by one or more processors, wherein the instruction, when executed, cause the one or more processors to perform operations comprising:
receiving, from a user device, image data of a meal associated with a user of the user device;
determining, based at least in part on the image data, one or more microbiome metrics associated with the meal;
determining, based at least in part on the one or more microbiome metrics associated with the meal, a visual characteristic associated with presenting an avatar on a display, the avatar representing a health of a microbiome of the user; and
causing the visual characteristic and the avatar to appear on the display.

16. The one or more non-transitory computer readable media of claim 15, wherein the operations further comprise:
determining, based at least in part on the consumption data, nutritional data associated with the meal; and
wherein determining the one or more microbiome metrics is based at least in part on the nutritional data.

17. The one or more non-transitory computer readable media of claim 16 wherein the operations further comprise:
logging the nutritional data and the meal; and
providing the user access to the logged nutritional data.

18. The one or more non-transitory computer readable media of claim of claim 15, wherein the operations further comprise:
receiving personalization data associated with the user; and
generating, based at least in part on the personalization data, the avatar.

19. The one or more non-transitory computer readable media of claim 18, wherein the personalization data includes a lab result of a test of the microbiome of the user.

20. The one or more non-transitory computer readable media of claim 15, wherein the operations further comprise:
determining, based at least in part on the one or more microbiome metrics, a recommended recipe for the user; and
causing the recommended recipe to be presented on a display of the user device.
